(19) United States
(12) Patent Application Publication
(10) Pub. No.: US 2008/0119492 A1
(43) Pub. Date: May 22, 2008

(54) Compositions and methods for treating cancer and other diseases characterized by abnormal cell proliferation

(76) Inventors: Jean-Marie Lehn, Strasbourg (FR); Jean-Louis Mergny, Villejuif (FR); Anne Petitjean, Kingston (CA); Maia-Paule Teulade-Fichou, Paris (FR); Andrei Tchirkov, Royat (FR); Pierre Verrelle, Chamalieres (FR)

Correspondence Address: KING & SPALDING LLP, 1180 PEACHTREE STREET, ATLANTA, GA, 30309-3521, US

(21) Appl. No.: 11/374,312

(22) Filed: Jul. 28, 2006

Related U.S. Application Data
(60) Provisional application No. 60/704,184, filed on Jul. 29, 2005.

(51) Int. Cl. A61K 31/506; C07D 403/14

(52) U.S. Cl. 514/256; 544/294

(57) ABSTRACT

The present invention relates to compositions and methods for treating cancer and other disease states characterized by abnormal cell proliferation. In particular, the present invention relates to certain novel non-nucleoside pharmacologically active compounds that alter telomere and telomerase functions, regulate cell proliferation and/or treat cancer and other diseases characterized by abnormal cell proliferation. The present invention also pertains to pharmaceutical compositions comprising such compounds, and the use of such compounds and compositions, both in vitro and in vivo, to inhibit telomerase, to regulate cell proliferation, and to treat cancer or to inhibit the proliferation of the rumor cells, and to treat other proliferative conditions.

### CROSS-REFERENCE TO RELATED APPLICATIONS

This patent application claims the benefit of U.S. Provisional Patent Application No. 60/704,184, filed Jul. 29, 2005, which is incorporated herein by reference in its entirety.

### FIELD OF THE INVENTION

This invention pertains generally to the field of chemistry and pharmaceuticals, and more specifically to certain novel non-nucleoside compounds that alter telomere and telomerase functions, regulate cell proliferation and/or treat cancer and other diseases characterized by abnormal cell proliferation. The present invention also pertains to pharmaceutical compositions comprising such compounds, and the use of such compounds and compositions, both in vitro and in vivo, to inhibit telomerase, to regulate cell proliferation, and to treat cancer or to inhibit the proliferation of the tumor cells, and to treat other proliferative conditions.

### BACKGROUND OF THE INVENTION

Cellular proliferation is a normal ongoing process in all living organisms and one that involves numerous factors and signals that are delicately balanced to maintain regular cellular cycles. Cellular proliferation is the culmination of a cell's progression through the cell cycle resulting in the division of one cell into two cells. The 5 major phases of the cell cycle are G₀, G₁, S, G₂ and M. During the G₀ phase, cells are quiescent. Most cells in the body, at any one time, are in this stage. During the G₁ phase, cells respond to signals to divide and produce the RNA and proteins necessary for DNA synthesis. During the S-phase (SE, early S-phase; SM, middle S-phase; and SL, late S-phase) the cells replicate their DNA. During the 32 phase, proteins are elaborated in preparation for cell division. During the mitotic (M) phase, the cell divides into two daughter cells. Because organisms are continually growing and replacing cells, cellular proliferation is a central process that is vital to the normal functioning of almost all biological processes. Mammalian cells are normally subject to rigid controls regulating the cell cycle in order to maintain the integrity of the function and structure of the whole organism. When normal cellular proliferation is disturbed or somehow disrupted, the results can affect an array of biological functions. Alterations in cell cycle progression occur in all cancers and may result from over-expression of genes, mutation of regulatory genes, or abrogation of DNA damage checkpoints (Hochhauser, 1997). Some disorders characterized by abnormal cellular proliferation include cancer, abnormal development of embryos, difficulty in wound healing, as well as malfunctioning of inflammatory and immune responses. Many other hyperproliferative disorders also exist. Hyperproliferative disorders are also caused by non-cancerous (i.e., non-neoplastic) cells that overproduce in response to repetitions in the cell cycle. Examples of such hyperproliferative disorders and benign growth disorders include, but are not limited to, diabetic retinopathy, psoriasis, lipomas and prostate enlargement.

A defining feature of cancer is abnormal cellular proliferation. Cancer cells respond abnormally to control mechanisms that regulate the division of normal cells. Cancer cells continue to divide in a relatively uncontrolled fashion until they harm and/or kill the host. Thus, cancer is an aberrant net accumulation of atypical cells, which can result from an excess of proliferation, an insufficiency of cell death, or a combination of the two.

It is clear that cellular proliferation plays a major role in the pathology and growth and development of cancer and metastases of cancer. One of the devastating aspects of cancer is the propensity of cells from malignant neoplasms to disseminate from their primary site to distant organs and develop into metastatic cancers. By the time that the primary tumor is identified and localized, seed cells often have escaped and migrated or metastasized to other organs in the body where they establish secondary tumors. Surgical procedures are rarely sufficient to cure a cancer because even after the primary tumor is removed, multiple secondary tumors survive and proliferate. Despite advances in surgical treatment of primary neoplasms and aggressive therapies, most cancer patients die as a result of metastatic disease. Hence, there is an immediate and pressing need for new and more efficacious cures for cancer.

As stated, compromise of any of the steps involved in cell cycle regulation may result in escape from regulatory mechanisms and lead to neoplasia. However, even if a cell escapes proliferation suppression, there are limitations to the number of replicative cycles a cell can progress through before safety mechanisms cause cessation of the cell cycle. Normal somatic cells undergo a limited number of cell divisions and then senescence. Cellular senescence is the programmed cell death response leading to growth arrest of cells (Dimri et al., 1995). DNA damage, exposure of colon, breast and ovarian cancer cells to topoisomerase inhibitors and exposure of nasopharyngeal cancer cells to cisplatin are reported to prevent proliferation of these cells by induction of senescence (Wang et al., 1998; Poele et al., 1999). A major candidate for the molecular signal for replicative senescence is that of telomere shortening.

Telomeres are nucleoprotein structures at the ends of linear chromosomes consisting of DNA sequences arranged in tandemly repeated units which extend from less than 100 to several thousands of bases. (Neidle et al., 2005) In humans, telomeres consist of the sequence 5′-TTAGGG (SEQ ID No. 1) (Blackburn, 1991; Blackburn et al., 1995). Telomere sequences vary from species to species, but are generally GC-rich. Telomeres have several functions apart from protecting the ends of chromosomes, the most important of which appear to be associated with senescence, replication, and the cell cycle clock (Counter et al., 1992). Progressive rounds of cell division result in a shortening of the telomeres by some 50-200 nucleotides per round. Research has established that almost all tumor cells have shortened telomeres, which are maintained at a constant length (Allshire et al., 1988; Harley et al., 1990; Harley et al., 1994) and are associated with chromosome instability and cell immortalization.

During each round of cellular replication, both strands of DNA separate and daughter strands are synthesized in a slightly different manner on the leading and lagging strand. While the lead strand replicates in a continuous fashion using conventional DNA polymerase, the lagging strand replicates in a discontinuous fashion using Okazaki fragments. Gaps between individual Okazaki fragments are filled in by the regular DNA polymerase. However, there remains a potential end replication problem, which arises because Okazaki fragment priming will not necessarily start at the very end of the DNA and because the RNA primer, once removed, would result in a portion of unreplicated 3′-DNA (an unrepaired 3′-overhang). This can lead to a loss of 50-200 base pairs with every round of somatic cell division, with eventual shortening of telomeres to a length that coincides with the activation of an anti-proliferative mechanism called “mortality stage 1” (M1). Senescence in somatic cells occurs during M1. Telomere shortening functions as a “mitotic clock” and limits the number of divisions in somatic cells to about 50-70 times, ultimately contributing to cell aging and apoptosis. (Neidle et al., 2005b)

In some cells, due to various mechanisms, the M1 stage is bypassed and cells can continue to divide until telomeres become critically shortened, called “mortality stage 2” (M2). At this M2 stage, in many immortalized cells, a specialized DNA polymerase called “telomerase” appears and utilizes its internal RNA template to synthesize the telomeric sequence and compensate for the loss of telomeric DNA due to incomplete replication. This prevents further shortening of telomeres, and the resulting stabilization of their length contributes to immortalization. (Neidle et al., 2005b)

Telomerase adds the telomeric repeat sequences onto telomere ends, ensuring the net maintenance of telomere length in tumor cells commensurate with successive rounds of cell division. A significant recent finding has been that approximately 85-90% of all human cancers are positive for telomerase, both in cultured tumor cells and primary tumor tissue, whereas most somatic cells appear to lack detectable levels of telomerase (Kim et al., 1994; Hiyama et al., 1995a). This finding has been extended to a wide range of human tumors (see, for example, references Broccoli, 1994 and Hiyama et al., 1995b) and is likely to be of use in diagnosis.

Thus, human telomerase has been proposed as a novel and potentially highly selective target for antitumor drug design (Feng et al., 1995; Rhyu et al., 1995; Parkinson, 1996). Telomerase is not normally expressed in most mammalian somatic cells. Exceptions to this rule include male germ line cells and some epithelial stem cells (e.g., as in the intestinal crypts, the basal layer of the epidermis, and within human hair follicles). The absence of telomerase in most normal cells makes this enzyme a particularly attractive target, considering that its inhibition may cause minimal damage to the whole patient.

As stated, both telomerase activity and shortened but stabilized telomeres have been detected in the majority of tumors examined (and in over 90% of all human cancers examined), and consequently, telomeres and telomerase are recognized targets for anti-neoplastic (e.g., cancer) chemotherapy. The fact that tumor cells have shorter telomeres and higher proliferation rates than normal replicative cell populations suggests that a therapeutic telomerase inhibitor may cause tumor cell death well before damage to regenerative tissues occurs, thereby minimizing undesirable side-effects. Therapies directed at control of the cellular proliferative processes, in particular, to the inhibition of telomerase activity, could lead to the abrogation or mitigation of cancer.

Further, studies with antisense constructs against telomerase RNA in HeLa cells show that telomere shortening is produced, together with the death of these otherwise immortal cells (Feng et al., 1995). Sequence-specific peptide-nucleic acids directed against telomerase RNA have also been found to exert an inhibitory effect on the enzyme (Norton et al., 1996).

For a more detailed discussion regarding telomeres and telomerase, and their role as anti-proliferative targets, see, for example, Sharma et al., 1997; Urquidi et al., 1998; Perry et al., 1998c; Autexier, 1999; Neidle et al., 1999; 2001; Rezler et al., 2002; Parkinson et al., 2002; Neidle et al., 2002a, Gowan et al., 2002; Herbert et al., 2001; Shay et al., 2002; Mergny et al., 2002; and Corey et al., 2002.

Recently, a range of telomerase inhibitors have been investigated. See, for example, Carrasco et al., 2002; Alberti et al., 2002; Riou et al., 2002; Kim et al., 2002; Kern et al., 2002; Gomez et al., 2002. In addition, a number of polycyclic compounds, including polycyclic acridines, anthraquinones, and fluorenones have been shown to inhibit telomerase and/or to have anti-tumor effects in vitro. See, for example, Bostock-Smith et al., 1999; Gimenez-Amau et al., 1998a; Gimenez-Amau et al., 1998b; Hagan et al., 1997; Hagan et al., 1998; Harrison et al., 1999; Julino et al., 1998; Perry et al., 1998a, 1998b, 1999a, 1999b; Sun et al., 1997. Neidle et al., 2002, describe various disubstituted acridines and trisubstituted acridines, which inhibit telomerase and/or regulate cell proliferation.

Most prior art anti-cancer therapies, whether directed to induction of cell cycle arrest, inhibition of proliferation, induction of apoptosis or stimulation of the immune system, have proven to be less than adequate for clinical applications. Many of these therapies are inefficient or toxic, have significant adverse side effects, result in development of drug resistance or immunosensitization, and are debilitating for the recipient.

The identification of therapeutic compounds which have modulation or inhibitory activity against human telomerase is a desirable goal, particularly compounds and methods of treatment of cancers in which telomerase contributes to the immortality of cells and undesirable or abnormal proliferation of cells. Toward this end, there have been efforts to identify compounds that affect telomerase activity. Thus, a goal of current medical research has been to understand and treat cellular disorders, and to selectively target cancer cells either by altering the telomere or inhibiting telomerase activity.

Although many are known, there remains an immediate need for effective telomerase inhibitors, anti-cancer agents, and anti-proliferative agents which demonstrate, among other advantages, improved activity; improved selectivity (e.g., against tumor cells versus normal cells); minimal or no side-effects; simpler methods of administration; reduction in dosage amounts and frequency of administration; ease of synthesis, purification, handling, and storage; as well as reduced cost of synthesis, purification, handling, storage; and which can be combined with other treatments (e.g., chemotherapeutic agents).

Particularly preferred telomerase inhibitors are ones which are characterized by one or more of the following properties:

(a) significant inhibition in the growth of cells with high telomerase expression;

(b) shortened telomere length in tumor cells and in human tumor xenografts;

(d) induction of an alteration in telomere-capping function, independently of telomere shortening or the level of telomerase expression;

(e) substantial increase in the percentage of tumor cells in the G2/M phase;

(f) radiosensitizing capabilities; and

(g) non-toxic and produce little to no side effects.

Thus, there is an immediate need for compounds which are telomerase inhibitors, anti-cancer agents, anti-proliferative agents, among others, which offer one or more of the above-listed advantages and properties.

What is needed, therefore, are compositions and methods which can inhibit abnormal or undesirable cellular proliferation related to tumors.

### SUMMARY OF THE INVENTION

This invention relates to compositions and methods for treating disease states characterized by abnormal cell proliferation. In particular, this invention relates to pharmacologically active compounds comprising novel non-nucleoside compounds which inhibit telomerase, regulate cell proliferation, and treat cancer or inhibit the proliferation of the tumor cells, and treat other hyperproliferative conditions. The present invention overcomes one or more of the problems inherent in the prior art by providing compositions and methods for use in the inhibition and modulation of telomerase activity. In particular, the present invention relates to a novel class of non-nucleoside molecules that are capable of inhibiting telomerase activity. These compounds have demonstrated their ability to interact with telomeres which form G-quadruplex structures. As telomeres are involved in controlling the cell cycle, cell replication and aging, these inhibitors of telomerase prevent uncontrolled cell growth and the immortality of tumor cells. Further, the present invention encompasses the synthesis and preparation of a novel guanine quadruplex binder and demonstrates the ability of the novel compounds to produce anti-growth and radiosensitization effects in cancer cells.

In search for new drugs with novel mechanisms of action, G-quadruplex-interactive agents are being considered, as they alter telomere and telomerase functions, thus inhibiting the proliferation of tumor cells. Since telomerase appears to be found almost exclusively in tumor cells, these agents are contemplated to be useful as antitumor agents. Compounds that inhibit telomerase are, therefore, potential drugs for the treatment of cancer.

Further, compounds such as those described here, which interact selectively with G-quadruplex structures and inhibit telomerase, are expected to be useful as inhibitors of the proliferation of cells that require telomerase to maintain telomere length for continued growth. The compounds of this invention were selected based upon their ability to interact with the human DNA-G-quadruplex structure. In addition, the present invention contemplates methods of treating cancer using the compounds in combination with radio- and chemotherapies.

Certain compounds within the scope of the general formulae set forth below and in the claims are useful for treating mammalian diseases characterized by abnormal cell proliferation or undesirable cell mitosis. Without wishing to be bound to any particular theory, it is believed that the compounds of the present invention are capable of fixing themselves to the O-quadruplex structure formed by the telomeres, thus inhibiting telomerase activity and reducing or inhibiting cell proliferation.

Mammalian diseases characterized by abnormal cell proliferation or undesirable cell mitosis, as defined herein, includes, but is not limited to, cancer, solid tumors and tumor metastases, excessive or abnormal stimulation of endothelial cells (e.g., atherosclerosis), benign tumors, (e.g., hemangiomas, acoustic neuromas, neurofibromas, trachomas, and pyogenic granulomas), vascular malfunctions, abnormal wound healing, inflammatory and immune disorders, Bechet's disease, gout or gouty arthritis, rheumatoid arthritis, psoriasis, diabetic retinopathy, and other ocular diseases such as retinopathy of prematurity (retrolental fibroplasic), macular degeneration, corneal graft rejection, and neovascular glaucoma, obesity, metabolic syndrome, diabetes, vascular and renal disorders, and pulmonary hypertension, and Osler Weber syndrome.

To be maximally effective, the compounds need to be formulated so that they are more bioavailable and more effective for their intended therapeutic use. The present invention addresses these formulation requirements.

One of the major challenges in oncology is the effective treatment a tumor, since tumors are often resistant to traditional therapies. The present invention contemplates combining the novel compounds with other therapeutic approaches such as, but not limited to, surgery and chemotherapy. It is also possible to combine a radiation treatment with the compounds of the present invention. These combination therapy may be administered simultaneously, separately or sequentially. The combination therapy is typically adapted by the practitioner to the patient in need of the treatment.

Accordingly, it is an object of the present invention to provide a composition and method for treating cancer and other disease states and conditions.

It is another object of the invention to provide a composition and method for inhibiting abnormal cell proliferation.

Yet another object of the invention relates to the preparation and use of pharmaceutical compositions having an anti-telomerase effect.

Another object of the invention pertains to active compounds, as described herein, which treat conditions which are known to be mediated by telomerase, or which are known to be treated by telomerase inhibitors.

A further object of the invention pertains to active compounds, as described herein, which (a) regulate (e.g., inhibit) cell proliferation; (b) inhibit cell cycle progression; (c) promote apoptosis; or (d) a combination of one or more of these.

Another object of the invention pertains to active compounds, as described herein, which are anti-telomerase agents, and which treat a condition mediated by telomerase.

Yet another object of the invention pertains to active compounds, as described herein, which are anticancer agents, and which treat cancer.

Another object of the invention pertains to active compounds, as described herein, which are antiproliferative agents, and which treat a proliferative condition.

A further object of the present invention pertains to a composition comprising compounds as described herein and a pharmaceutically acceptable carrier.

Another object of the present invention pertains to methods of inhibiting telomerase in a cell, comprising contacting said cell with an effective amount of the active compounds, as described herein.

Another object of the present invention pertains to methods of (a) regulating (e.g., inhibiting) cell proliferation; (b) inhibiting cell cycle progression; (c) promoting apoptosis; or (d) a combination of one or more of these, comprising contacting a cell with an effective amount of the active compounds, as described herein, whether in vitro or in vivo.

Another object of the present invention pertains to methods of treating a condition which is known to be mediated by telomerase, or which is known to be treated by telomerase inhibitors, comprising administering to a subject in need of treatment a therapeutically-effective amount of the active compounds, as described herein.

Another object of the present invention pertains to methods of treating cancer, comprising administering to a subject in need of treatment a therapeutically-effective amount of the active compounds, as described herein.

Another object of the present invention pertains to methods of treating a proliferative condition comprising administering to a subject in need of treatment a therapeutically-effective amount of the active compounds, as described herein.

Another object of the present invention pertains to the active compounds, as described herein, for use in a method of treatment of a human or animal by therapy.

Another object of the present invention pertains to use of the active compounds, as described herein, for the manufacture of a medicament for use in the treatment of a condition mediated by telomerase, cancer, a proliferative condition, or other condition as described herein.

Another object of the present invention pertains to a kit comprising (a) the active compounds, preferably provided as a pharmaceutical composition and in a suitable container and/or with suitable packaging; and (b) instructions for use, for example, written instructions on how to administer the active compounds.

Another object of the present invention pertains to compounds obtained by a method of synthesis as described herein, or a method comprising a method of synthesis as described herein.

Another object of the present invention pertains to novel intermediates, as described herein, which are suitable for use in the methods of synthesis described herein.

Another object of the present invention pertains to the use of such novel intermediates, as described herein, in the methods of synthesis described herein.

A further object of the invention is to provide a composition and method for enhancing radiation sensitivity of rumors.

A further object of the invention is to provide a simple and easily administered, preferably orally, composition that is capable of treating cancer.

These and other objects, features and advantages of the invention will be apparent from the following description of preferred embodiments thereof. As will be appreciated by one of skill in the art, features and preferred embodiments of one aspect of the invention will also pertain to other aspects of the invention.

### BRIEF DESCRIPTION OF THE DRAWINGS

FIG. 1 depicts the general structure of the compounds of the invention.

FIG. 2 demonstrates the effect of the composition on glioblastoma multiforme (GBM) cell proliferation.

FIG. 3 shows partial metaphase of a SF 763 cell showing telomere fusions. The fusion frequency was 0.3 in untreated cells and 1.6 in ThAcr2-treated cells.

FIG. 4 demonstrates clonogenic survival of GMB cells treated with combination of ThAcr2 and radiation. Survival of cells having combination therapy is markedly decreased as compared with survival after radiation along (P<0.001).

### DETAILED DESCRIPTION OF THE INVENTION

The present invention may be understood more readily by reference to the following detailed description of specific embodiments included herein. Although the present invention has been described with reference to specific details of certain embodiments thereof, it is not intended that such details should be regarded as limitations upon the scope of the invention. The entire text of the references mentioned herein are hereby incorporated by reference in their entireties.

This invention comprises certain novel non-nucleoside compounds that alter telomere and telomerase functions, regulate cell proliferation and/or treat cancer and other diseases characterized by abnormal cell proliferation. The present invention also comprises pharmaceutical compositions including, but not limited to, such compounds, and the use of such compounds and compositions, both in vitro and in vivo, to inhibit telomerase, to regulate cell proliferation, and to treat cancer or inhibit the proliferation of the tumor cells, and to treat other proliferative conditions.

Throughout this specification, including any claims which follow, unless the context requires otherwise, the word “comprise,” and variations such as “comprises” and “comprising,” will be understood to imply the inclusion of a stated integer or step or group of integers or steps, but not the exclusion of any other integer or step or group of integers or steps.

It must be noted that, as used in the specification and any appended claims, the singular forms “a,” “an,” and “the” include the plural unless the context is inappropriate. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

The term “effective amount” is meant a therapeutically or prophylactically effective amount. Such amounts can be readily determined by an appropriately skilled person, taking into account the condition to be treated, the route of administration and other relevant factors. Such a person will readily be able to determine a suitable dose, mode and frequency of administration.

As used herein, the term “soluble” means partially or completely dissolved in an aqueous solution.

The term “biological activity” refers to the functionality, reactivity, and specificity of compounds that are derived from biological systems or those compounds that are reactive to them, or other compounds that mimic the functionality, reactivity, and specificity of these compounds.

The term “telomerase inhibitor” refers to a compound, molecule, reagent, drug, or chemical which is capable of decreasing the activity of the telomerase enzyme in vitro or in vivo. Such inhibitors can be readily identified using standard screening protocols in which a cellular extract or other preparation having telomerase activity is placed in contact with a potential inhibitor, and the level of telomerase activity measured in the presence or absence of the inhibitor, or in the presence of varying amounts of inhibitor.

As described below, compounds that are useful in accordance with the invention include novel molecules that are telomerase inhibitors.

In addition to single drug activity, the compounds of the present invention inhibit the activity of telomerase and are capable of enhancing sensitivity to ionizing radiation. U-shaped molecules holding two potentially intercalating units 7 Å apart have been designed to have a moderate affinity for duplex DNA on the basis of the nearest neighbor exclusion principle but may display strong binding affinity for other architectures such as guanine quadruplexes. Organic cations such as {ThAcr₂Me⁺+ThAcr₂Me₂²⁺} produced as a 1:2 mixture of mono- and di-cation species, have a strong affinity for model guanine quadruplexes inspired from human telomeres.

In the present text, “ThAcr2” refers to a 12 mixture of ThAcr₂Me⁺, TfO⁻ and ThAcr₂Me₂²⁺, 2TfO⁻. ThAcr₂ (I) refers to ThAcr₂Me⁺, Cl⁻ and ThAcr2 (II) refers to ThAcr₂Me₂²⁺, 2Cl⁻.

The present invention encompasses compounds having a general spacer structure of A-R-A, wherein A represents heterocyclic groups, preferably cationic heterocycles derived from pyridine derivatives such as acridinium groups, wherein R is a hydrocarbon or heterocatomic spacer, preferably a heterocyclic sequence such as a substituted pyridine-pyrimidine-pyridine (py-pym-py) or pyridine-pyridine-pyridine (py-py-py) or pyridine-hydrazone-pyridine (py-hyz-py) sequence, and wherein R₁ is a hydrogen, an aliphatic or an aromatic substituent. The structure of the spacer is as follows

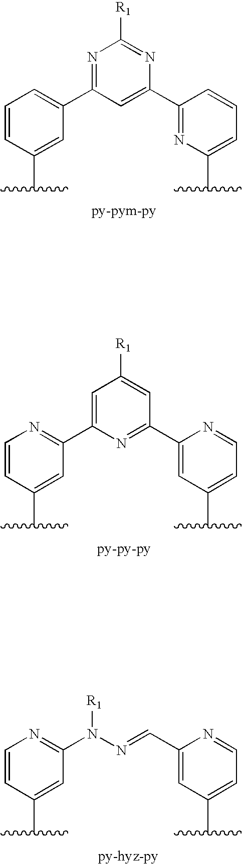

The heterocyclic molecules of the present invention are constructed on an oligoheterocyclic spacer such as the pyridine-pyrimidine-pyridine scaffold, bearing two intercalating units (cf A and B) as follows:

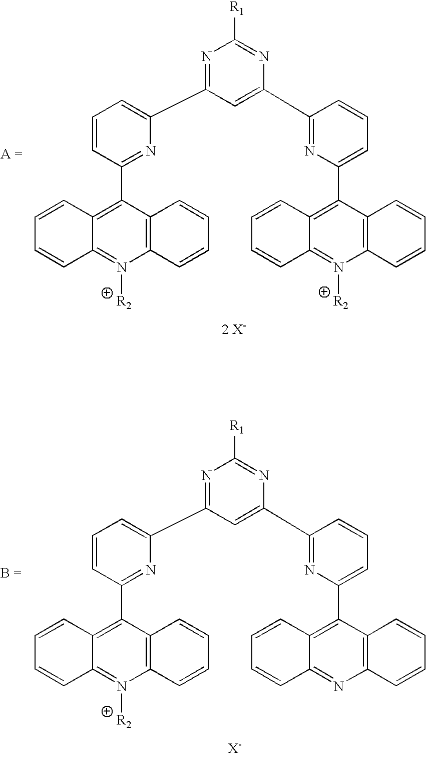

wherein R₁ is hydrogen, or an aliphatic, aromatic, hydrophilic, hydrophobic, hydrophilic, cyclic, acyclic substituent; and wherein R₂ is an alkyl, hydrophilic, pr hydrophobic substituent; and wherein X⁻ (or 2X⁻) are anions such as triflate, chloride, bromide, iodide, nitrate, sulphate.

The present invention describes the synthesis and preparation of cationic N-acridyl alkylated 4,6-bis-(6-(acrid-9-yl)-pyridin-2-yl)-pyrimidine, and demonstrates the activity of such species on cancer cells as single agents as well as synergistic effects in combination with ionizing radiations.

The compounds of the present invention comprise chemotherapeutic agents and may be combined with other therapies, such as radiosurgery, radiotherapy and other chemotherapeutic agents. Often combining a host of different treatment methods proves more effective in cancer therapy than using one treatment method alone. It is contemplated to use the novel compounds of the present invention as chemotherapeutic agents in addition to several other well known chemotherapeutic agents.

(a) Chemotherapy: A variety of chemical compounds, described as chemotherapeutic agents, are used to treat tumors. Chemotherapeutic agents contemplated to be of use with the present invention include, but are not limited to, adriamycin, 5-fluorouracil (5-FU), etoposide (VP-16), camptothecin, actinomycin-D, mitomycin, cisplatin (CDDP), hydrogen cyclophosphamide, ifosfamide, melphalan, chlorambucil, bisulfan, nitrosurea, dactinomycin, daunorubicin, doxorubicin, bleomycin, plicomycin, tamoxifen, taxol, transplatinum, vincristin, vinblastin, and methotrexate. Chemotherapeutic agents capable of inducing damage to DNA include compounds that interfere with the replication, mitosis, or chromosomal segregation of DNA. Such agents include, but are not limited to, adriamycin, doxorubicin, etoposide, verapamil, and podophyllotoxin. The novel compounds of the present invention are capable of inhibiting telomerase activity by interacting with the G-quadruplex structure of the telomere. The novel compounds of the present invention are cytotoxic because of their capability of inhibiting telomerase activity, which is critical for cell replication and maintenance of cancer cell immortality. In the combination therapy contemplated by the present invention, one would generally contact the target cells with the novel compounds of the present invention and at least one other chemotherapeutic agent mentioned herein. The combination of the compounds and at least one other chemotherapeutic agent would be administered in an effective amount sufficient to kill the cell, or inhibit the proliferation of the cell. This combination treatment may also be achieved by contacting the target cells with a single composition or pharmacological formulation that includes both agents, or by contacting the target cells with one or more distinct compositions or formulations, either sequentially, simultaneously or intermittently.

(b) Radiotherapy: The compounds of the present invention are capable of radiosensitizing cancer cells and may be used in combination with external, internal and systematic radiations as well as hyperthermia, radio-immunotherapy and photodynamic therapy, with X-ray radiations, and gamma-ray radiations, as well as particle beams. Radiotherapeutic agents include radiation and waves that induce DNA damage, including, but not limited to, X-rays, γ-irradiation, UV-irradiation, microwaves, electronic emissions, and radioisotopes. Therapy may be achieved by irradiating the localized tumor site with the types of radiations mentioned herein. It is believed that all of the radiotherapeutic agents effect a broad range of DNA, the precursors of DNA, the replication and repair of DNA, and the assembly and maintenance of chromosomes. Dosage ranges for X-rays range from daily doses of 50 to 200 roentgens for prolonged periods (3 to 4 weeks), to single doses of 2000 to 6000 roentgens. Dosages ranges for radioisotopes vary widely, and depend on the half-life of the isotope, the strength and type of radiation emitted, and the uptake of radiation by the tumor cells. The compounds of the present invention may also be utilized in prophylactic radiotherapy and palliative radiotherapy. The compounds of the present invention may also be used in combination with radiomimetic agents including, but not limited to, bleomycin, neocarzinostatin, adriamycin, mitomycin C, streptonigrin, methylmethanesulfonate and enediynes.

(c) Surgery and radiosurgery. Surgical treatment is a traditional procedure utilized to remove a portion of or the entire cancerous growth or tumor. Surgery is often combined with radiotherapy or chemotherapy to ensure the destruction of any remaining neoplastic or malignant cells.

Specific compounds according to the invention are described below. The following is a general synthetic strategy and scheme for producing the novel compounds of the present invention. The compounds of the present invention were prepared in four steps starting from commercially available material and using the following synthetic route:

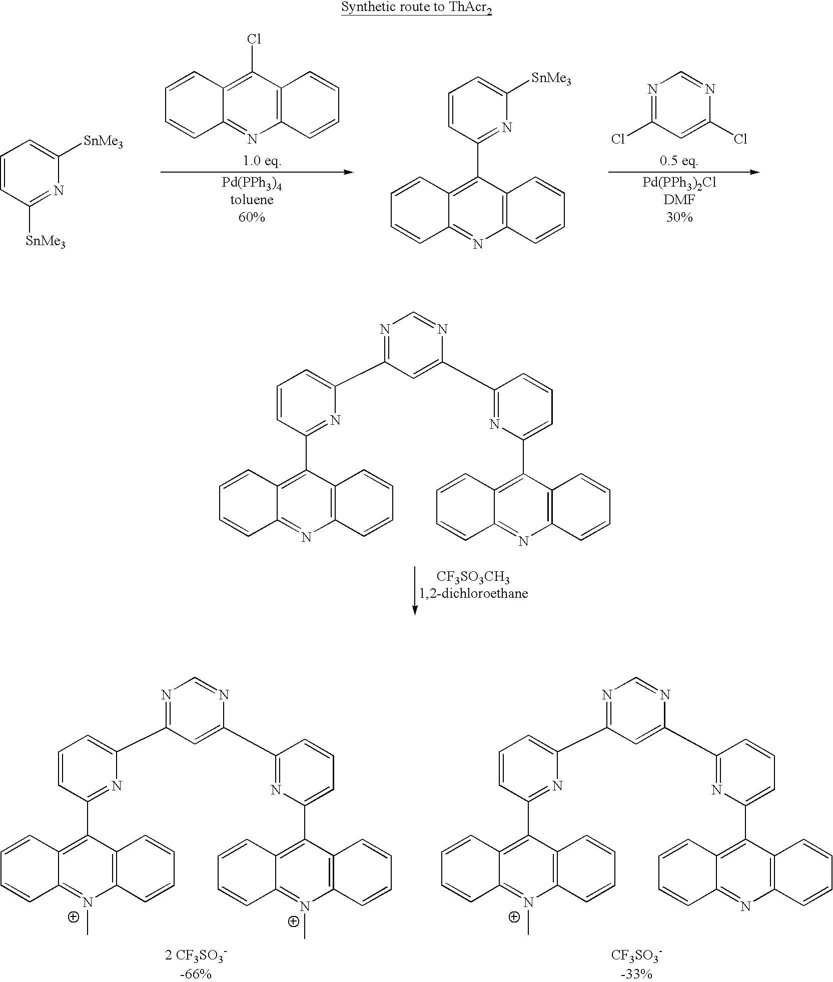

2,6-bis-(trimethylstannyl)pyridine was coupled with 9-chloroacridine under Stille coupling conditions to yield the monocoupled product in a 60% yield. The monocoupled product was reacted with 2,6-dichloropyrimidine under Stille conditions to afford the product of di-coupling in a 30% yield. Methylation of the acridine units was performed with methyltrifluoromethane sulfonate in dry dichloroethane and the product isolated as a triflate salt. The end product had sufficient water solubility to be used in the in vitro studies. ¹H NMR and HPLC analyses both indicated a 2:1 mono-bismethylated product distribution.

All commercially available reagents were used as received. 2,6-dichloropyridine, 4,6-dichloropyrimidine, 1,2-dichloroethane and methyltrifluoromethanesulfonate were obtained from Aldrich, sodium was purchased from Lancaster. 1,2-dichloroethane and toluene were dried on calcium dichloride and sodium respectively, and distilled at ambient pressure under argon. 2,6-bis-(trimethylstannyl)pyridine and 9-chloroacridine were prepared following procedures from the literature (Yamamoto et al., 1982). Products were characterized by ¹H and ¹³C NMR (when solubility permitted) using the residual signal from the solvent as a reference (tBuOH in D₂O), mass spectrometry (performed in the Laboratoire de Spectrométrie de masse Bio-organique, LSMBO, Strasbourg, France) and elemental analyses (performed by the elemental analysis services of Louis Pasteur University, Strasbourg, France).

9-(6-Trimethylstannyl-pyridin-2-yl)-acridine: To 4.9 mmol of 2,6-bis-(trimethylstannyl)pyridine solubilized in 32 mL of freshly distilled toluene were added 1.05 g of 9-chloroacridine (4.9 mmol, 1.0 eq.) and 288 mg of tetrakistriphenylphosphinepalladium (0.25 mmol, 0.05 eq.). The solution was purged with argon, protected from light, and refluxed under argon. Additional 100 mg of catalyst were added after 12 hours, and the solution refluxed for an additional 5 hours. After the solution was allowed to acclimate to room temperature, the mixture was evaporated onto basic alumina (activity 1), run through a short alumina column (hexane/Et₂O 3.0:0.2, R_{f}=0.34) and concentrated in vacuo to give a yellow solid (yield: 60%).

4,6-bis-6-acrid-9-yl)-pyridin-2-yl)-pyrimidine: To 3.2 mmol of 9-(6-trimethylstannyl-pyridin-2-yl)-acridine (2.24 eq.) suspended in 15 mL of DMF were added 227 mg of 4,6-dichloropyrimidine (1.52 mmol) and 53 mg of dichlorobistriphenyl phosphinepalladium (II) (7.55 10⁻⁵ mol, 0.05 eq.). The solution was purged with argon, protected from light and heated at 90° C. under argon for 18 hours. After concentration under vacuum, the brown solid was taken up in chloroform (320 mL) and washed with water (50 mL) and brine (50 mL). The organic layer was dried (Na₂SO₄), filtered, concentrated and the residue taken up in 40 mL of a 75:25 MeOH/Et₂O mixture, filtered and washed with Et₂O. The light brown solid was adsorbed onto basic alumina and purified by column chromatography (CHCl₃/EtOAc 2.0:0.5 to 1:1 and CHCl₃/MeOH 3.0:0.2) to give the desired product as a light yellow powder (360 mg; 30%). ¹H NMR (200 MHz, CDCl₃): 9.47 (d, J=1.2 Hz, 1H), 9.17 (d, J=1.2 Hz, 1H), 8.62 (dd, ³J=7.6 Hz, 2H), 8.24 (d, ³J=8.5 Hz, 4H), 8.11 (t, ³J=7.8 Hz, 2H), 7.55-7.7 (m, 10H), 7.15 (td, ³J=7.9 Hz, 4H), ¹³C NMR (200 MHz, CDCl₃): 164.1, 155.5, 154.7, 154.3, 148.8, 145.1, 143.8, 137.5, 129.9, 129.7, 128.0, 125.9, 124.5, 121.2, 115.3; R_{f} (SiO₂, CH₂Cl₂/CH₃OH 2.0:0.2)=0.20; mp>260° C.; FAB+: 589.1 (MH⁺); EA: C₄₀H₂₄N₆.0.24CHCl₃, calc.: % C=78.29, % H=3.96, % N=13.61, obs.: % C=78.30, % H=3.92, % N=13.71; UV-Vis (CH₂Cl₂): λₘₐₓ abs (log₁₀ε): 252 (5.3); 293 sh. (4.3), 348 (4.2), 361 (4.3), 384 (4.0); Fluo (CH₂Cl₂): λₘₐₓ em=440 nm.

4,6-bis-(6-(10-N-methylacrid-9-ylinium)pyridin-2-yl)-pyrimidine bistriflate (TAcr2): To 39 mg of bisacridine 4,6-bis-(6-(acrid-9-yl)-pyridin-2-yl)-pyrimidine (5.0 10⁻⁵ mol) solubilized in 25 mL of hot dry 1,2-dichloroethane, 20 μL of methyltrifluoromethanesulfonate was added under argon. The yellow solution was refluxed for 4 hours, then 5 μL of additional methyltrifluoromethanesulfonate was added and heating continued for an additional 2 hours. After the solution was allowed to cool to room temperature, the yellow precipitate was filtered and washed twice with diethylether (2 mL) and dried in vacuo to yield 45 mg of a bright yellow solid. ¹H NMR (400 MHz, D₂O): ˜2:1 mixture of bis- and mono-methylated 8.81 (s, 1H, mono), 8.77 (s, 1H, bis), 8.60 (d, ³J=9.2 Hz, 4H, mono), 8.54 (s, 1H, mono), 8.52 (s, 1H, bis), 8.05-8.3 (m, 12H, mono and bis), 7.78 (t, ³J=7.9 Hz, 2H, bis), 7.77 (t, ³J=7.7 Hz, 2H, mono), 7.3-7.45 (m, 16H, mono and bis), 7.05-7.15 (m, 5H, mono and bis), 4.93 (s, 6H, bis), 4.74 (s, 3H, mono); (HR)-FAB+: Bis: (C₄₂H₃₀N₆.CF₃SO₃): calc.: 767.2052; obs.: 767.2076; Mono (C₄₁H₂₇N₆)⁺: calc.: 603.7; obs: 603.1.

Additional compounds according to the invention are described below. The following is a general synthetic strategy and scheme for producing the novel compounds of the present invention.

### 1) ThAcr₂Mt₂²⁺, 2Cl⁻: ThAcr2 (II) in following scheme

### 2) ThAcr₂Me⁺, Cl⁻: ThAcr2 (I) in following scheme

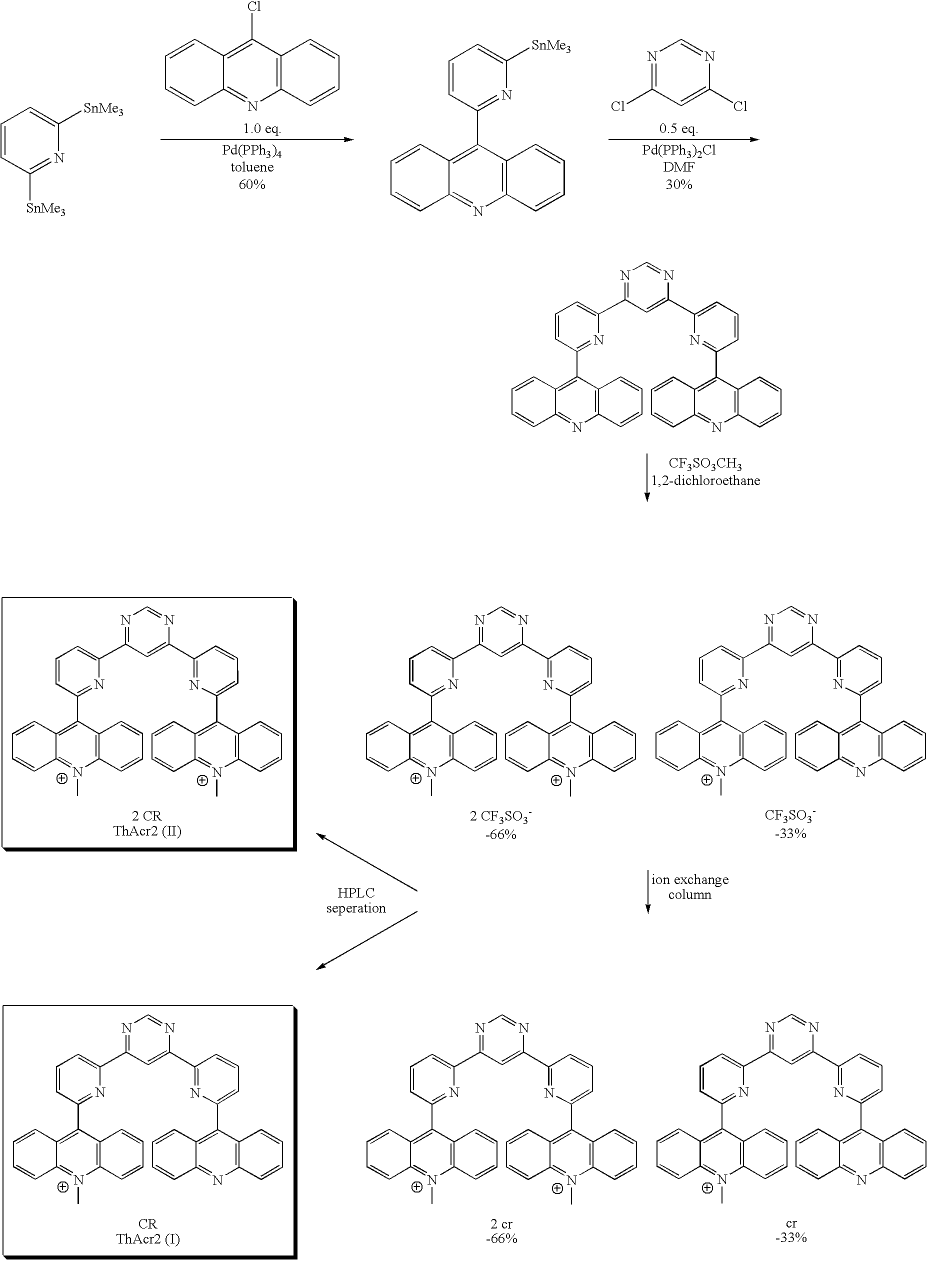

The ion exchange was performed using the following conditions: the mixture of the two alkylated products were solubilized in a minimum amount to 1:1 mixture of EtOH and water, then loaded onto a Amberlite IRA 400 ion exchange column conditioned with chloride anions. The yellow band (mixture of two products) was eluted with water and lyophilized.

ThAcr2 (1) and ThAcr2 (II) were separated by HPLC under the following conditions:
PRONTOSIL C4 column
Injection in water
gradient of two solvents (solvent A=acetonitrile and solvent B=10:90:10 acetonitrile/water/trifluoroacetic acid)
ramp: 0-20 minutes: 10% B to 70% B
   20-20.1 minutes: 70% B to 95% B
   25-25.1 minutes: 95% B to 10% B

The retention times in these conditions were 9 minutes for ThAcr2 (I) and 13 minutes for ThAcr2 (II).

### 3) TArAcr2 (II)

Synthesis: The synthesis of TArAcr₂Me₂²⁺, 2TfO⁻ is described in “Dynamic Devices. Shape Switching and Substrate Binding in Ion Controlled Nanomechanical Molecular Tweezers”, A. Petijean, J.-M. Lehn, R. G. Khoury, N. Kyritsakas; J. Am. Chem. Soc. 2004, 126, 6637-6647.

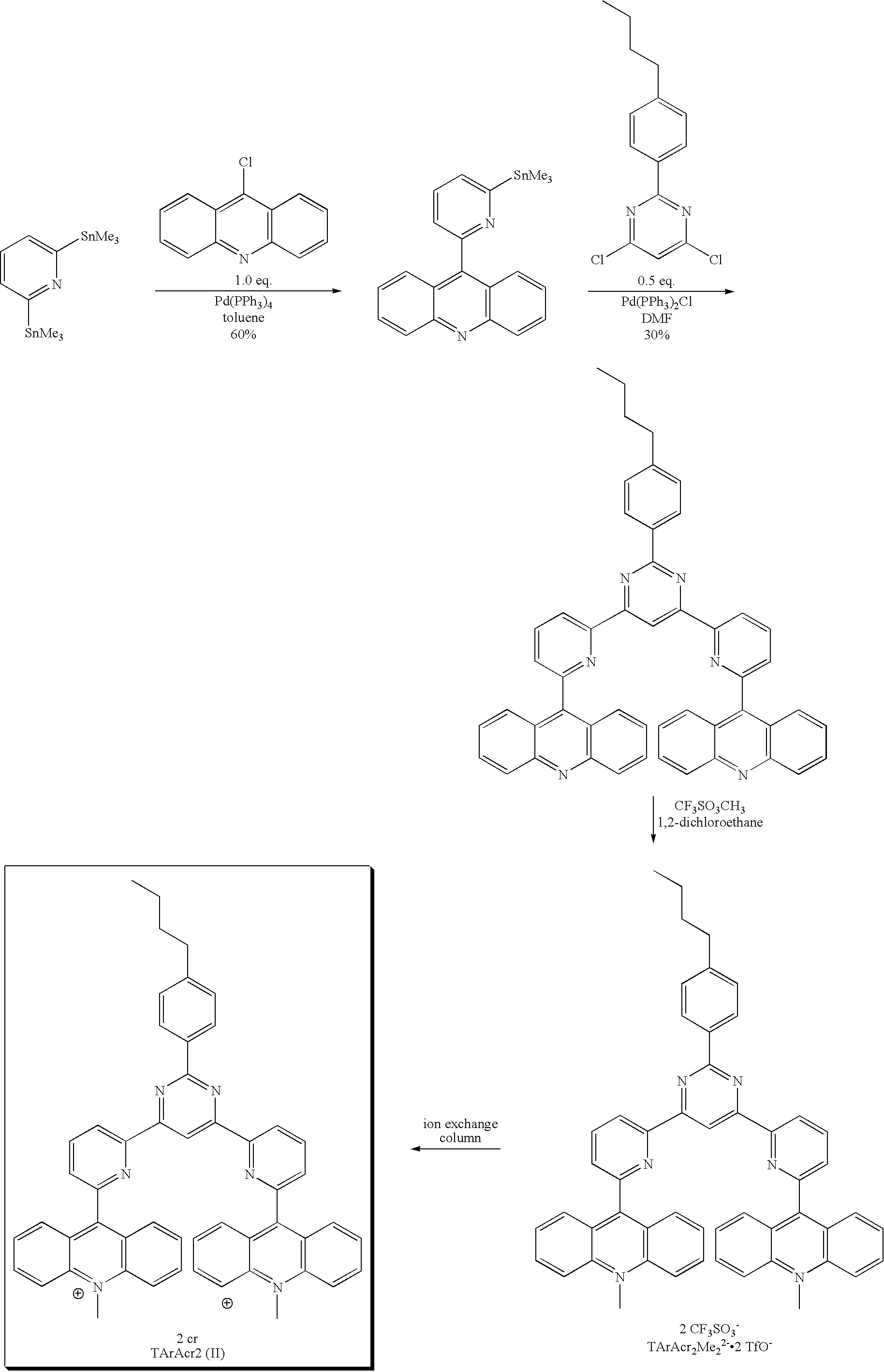

The ion exchange to TArAcr2 (11) was performed as described above. The yellow water solution was lyophilized. No HPLC purification was necessary as the bisalkylated product was formed quantitatively without contamination by the monoalkylated species.

Chemical names:
ThAcr2 (II): 4,6-bis-(6-(N-methylacridin-9-ium)-pyridin-2-yl)-pyrimidine bis-chloride.
ThAcr2 (I): 9-{6-(6-(6-Acridin-9-yl-pyridin-2-yl)-pyrimidin-4-yl]-pyridin-2-yl}-10-methyl-acridinium chloride.
TArAcr2 (II): 2-(4-n-butylphenyl)-4,6-bis-(6-(N-methylacridin-9-ium)-pyridin-2-yl)-pyrimidine bis-chloride.

To one skilled in the art, there are multiple alternative synthetic paths that can be used to prepare the compounds of the present invention. While the synthetic pathways discussed above have been put to practice, these paths are not the only viable routes available to one skilled in the art.

Known compounds that are used in accordance with the invention and precursors to novel compounds according to the invention can be purchased. Other compounds according to the invention can be synthesized according to known methods from publicly available precursors.

Isomers are contemplated by this invention, however a stereochemical isomer (labeled as α or β, or as R or S) may be a mixture of both in any ratio, where it is chemically possible by one skilled in the art. Also contemplated by this invention are both classical and non-classical bioisosteric atom and substituent replacements, such as are described by Patani et al. (1996) and are well known to one skilled in the art. Such bioisosteric replacements include, for example, but are not limited to, substitution of ═S or ═NH for ═O.

Those skilled in the art will appreciate that the invention extends to other compounds within the formulae given in the claims below, having the described characteristics. These characteristics can be determined for each test compound using the assays detailed below and elsewhere in the literature.

In one embodiment, the administration of the present novel compositions comprising ThAcr2, resulted in a reduction of glioblastoma cells.

### Administration and Dosage

The compounds of the present invention can be provided as physiologically acceptable formulations using known techniques, and these formulations can be administered by standard routes. In general, the combinations may be administered by the topical, oral, rectal or parenteral (e.g., intravenous, subcutaneous or intramuscular) route. In addition, the combinations may be incorporated into biodegradable polymers allowing for sustained release, the polymers being implanted in the vicinity of where delivery is desired, for example, at the site of a tumor. The biodegradable polymers and their use are described in detail in Brem et al. (1991).

Formulations contemplated as part of the present invention include, but are not limited to, nanoparticle formulations made by numerous methods as disclosed in U.S. patent application Ser. No. 10/392,403 (Publication No. US 2004/0033267), U.S. patent application Ser. No. 10/412,669 (Publication No. US 2003/0219490), U.S. Pat. No. 5,494,683, U.S. patent application Ser. No. 10/878,623 (Publication No. US 2005/0008707), U.S. Pat. No. 5,510,118, U.S. Pat. No. 5,524,270, U.S. Pat. No. 5,145,684, U.S. Pat. No. 5,399,363, U.S. Pat. No. 5,518,187, U.S. Pat. No. 5,862,999, U.S. Pat. No. 5,718,388, and U.S. Pat. No. 6,267,989, all of which are hereby incorporated herein by reference in there entirety. A review of drug formulation technology is “Water Insoluble Drug Formulation” by Rong Liu, editor, pp. 1-633, (2000) CRC Press LLC, which is incorporated herein by reference in its entirety.

By forming nanoparticles, the compositions disclosed herein may have increased bioavailability. Preferably, the particles of the compounds of the present invention have an effective average particle size of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods well known to those of ordinary skill in the art, it is understood that the particle sizes are average particle sires and the actual particle sizes will vary in any particular formulation. Often, surface stabilizers are used to form stable nanoparticles; however, this method of forming nanoparticles is only one of many different methods of forming effective nanoparticle compositions.

The dosage of the composition will depend on the condition being treated, the particular derivative used, and other clinical factors such as weight and condition of the patient and the route of administration of the compounds. However, for oral administration to humans, a dosage of 0.01 to 100 mg/kg/day, preferably 0.01-1 mg/kg/day, is generally sufficient.

The formulations include those suitable for oral, rectal, nasal, topical (including dermal, transdermal, buccal and sublingual), vaginal or parenteral (including subcutaneous, intramuscular, intravenous, intradermal, intraocular, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into associate the active ingredient with liquid carriers or finely divided solid carriers or both, and then, if necessary, shaping the product Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tables may be made by molding, in a suitable machine, a mixture of the powdered compounds moistened with an inert liquid diluent. The tablets may be optionally coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such as carriers as are known in the art to be appropriate.

Formulation suitable for inhalation may be presented as mists, dusts, powders or spray formulations containing, in addition to the active ingredient, ingredients such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tables of the kind previously described.

Preferred unit dosage formulations are those containing a daily dose or unit, daily sub-dose, as herein above recited, or an appropriate fraction thereof, of the administered ingredient.

It should be understood that in addition to the ingredients, particularly mentioned above, the formulations of this invention may include other agents convention in the art having regard to the type of formulation in question, for example, those suitable for oral administration may include flavoring agents.

It may be convenient or desirable to prepare, purify or handle the active compounds in the form of a prodrug. The term “prodrug”, as used herein, pertains to a compound which, when metabolized (e.g., in vivo), yields the desired active compounds. Typically, the prodrug is inactive, or less active than the active compounds, but may provide advantageous handling, administration, or metabolic properties. Accordingly, the present invention relates to conjugated prodrugs and uses thereof. More particularly, the invention relates to conjugates of the compounds, and the use of such conjugates in the prophylaxis or treatment of conditions associated with abnormal cell proliferation or accelerated cell division, such as cancer, and inflammatory conditions, such as asthma and rheumatoid arthritis and hyperproliferative skin disorders including psoriasis. The invention also relates to compositions including the prodrugs of the present invention and methods of synthesizing the prodrugs. In one aspect, the present invention provides a conjugated prodrug conjugated to a biological activity modifying agent. The incorporation of the compounds into a disease-dependently activated prodrugs enables significant improvement of potency and selectivity of this anti-cancer agent.

Also contemplated by the present invention are implants or other devices comprised of the compounds, drugs or prodrugs thereof wherein the drug or prodrug is formulated in a biodegradable or non-biodegradable polymer for sustained release. Non-biodegradable polymers release the drug in a controlled fashion through physical or mechanical processes without the polymer itself being degraded. Biodegradable polymers are designed to gradually be hydrolyzed or solubilized by natural processes in the body, allowing gradual release of the admixed drug or prodrug. The drug or prodrug can be chemically linked to the polymer or can be incorporated into the polymer by admixture. Both biodegradable and non-biodegradable polymers and the process by which drugs are incorporated into the polymers for controlled release are well known to those skilled in the art. Examples of such polymers can be found in many references, such as Brem et al., 199). These implants or devices can be implanted in the vicinity where delivery is desired, for example, at the site of a tumor.

In addition to the compounds of the present invention, the pharmaceutical composition of this invention may also contain, or be co-administered (simultaneously or sequentially) with, one or more pharmacological agents of value in treating one or more disease conditions referred to hereinabove.

A person skilled in the art will be able by reference to standard texts, such as Remington's Pharmaceutical Sciences 17th edition, to determine how the formulations are to be made and how these may be administered.

In a further aspect of the present invention there is provided use of compounds or prodrugs thereof according to the present invention for the preparation of a medicament for the prophylaxis or treatment of conditions associated with abnormal cell proliferation or accelerated cell division.

In a further aspect of the present invention there is provided a pharmaceutical composition comprising compounds or prodrugs thereof according to the present invention, together with a pharmaceutically acceptable carrier, diluent or excipient.

The pharmaceutical composition may be used for the prophylaxis or treatment of conditions associated with accelerated cell division or abnormal cell proliferation.

In a still further aspect of the present invention there is provided a method of prophylaxis or treatment of a condition associated with abnormal cell proliferation or accelerated or increased amounts of cell division, hypertrophic growth or inflammation, said method including administering to a patient in need of such prophylaxis or treatment an effective amount of the compounds or prodrugs thereof according to the present invention, as described herein. It should be understood that prophylaxis or treatment of said condition includes amelioration of said condition.

Pharmaceutically acceptable salts of the compounds may be prepared in any conventional manner, for example, from the free base and acid.

### Conditions to be Treated

The compositions and methods described herein are useful for treating human and animal diseases and processes characterized by abnormal cellular proliferation or undesirable cell mitosis. Such diseases include, but are not limited to, cancer, solid tumors and tumor metastases, abnormal stimulation of endothelial cells (e.g., atherosclerosis), benign tumors, (e.g., hemangiomas, acoustic neuromas, neurofibromas, trachomas, and pyogenic granulomas), vascular malfunctions, abnormal wound healing, inflammatory and immune disorders, Bechet's disease, gout or gouty arthritis, rheumatoid arthritis, psoriasis, diabetic retinopathy, and other ocular diseases such as retinopathy of prematurity (retrolental fibroplasic), macular degeneration, corneal graft rejection, neuroscular glaucoma, and Osler Weber syndrome (Osler-Weber-Rendu disease).

The present invention is especially useful in treating cancers including, but not limited to, any one or combination of brain cancer, in particular, glioblastoma multiforme, breast cancer, cancers of the head, neck or larynx, prostrate cancer, renal cell cancer, ovarian cancer, colon cancer, bladder cancer, pancreatic cancer, stomach cancer, esophageal cancer, cutaneous melanoma, liver cancer, small cell and non-small cell lung cancer, testicular cancer, kidney cancer, bladder cancer, cervical cancer, parathyroid cancer, penile cancer, rectal cancer, anal cancer, small intestine cancer, thyroid cancer, uterine cancer, Hodgkin's lymphoma, non-Hodgkin's lymphoma, lip and oral cancer, skin cancer, leukemia, spinal cancer, vaginal cancer, cancer of the vulva, soft tissue sarcomas, lymphoma, multiple myeloma, rhabdomyosarcoma, retinoblastoma, Ewing sarcoma, neuroblastoma, and osteosarcoma. Glioblastoma multiforme is the most common brain tumor in humans and is highly resistant to radio- and chemotherapy.

Diseases associated with neovascularization can be treated according to the present invention. Such diseases include, but are not limited to, ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyartcritis, Wegener's sarcoidosis, Scleritis, Steven-Johnson disease, pemphigoid, radial keratotomy, and corneal graph rejection.

Other diseases associated with neovascularization can be treated according to the present invention. Such diseases include, but are not limited to, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, and post-laser complications. Other diseases include, but are not limited to, diseases associated with rubeosis (neovascularization of the iris and the angle) and diseases caused by the abnormal proliferation of fibrovascular or fibrous tissue including all forms of proliferative vitreoretinopathy, whether or not associated with diabetes.

As mentioned above, another disease that can be treated according to the present invention is rheumatoid arthritis. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Other diseases characterized by abnormal cell proliferation that can be treated according to the present invention are hereditary hemorrhagic telangiectasia, osteoarthritis, chronic inflammation, Crohn's disease, ulcerative colitis, Bartonellosis, inflammatory or immune mediated bowel disease and acquired immune deficiency syndrome.

The present invention can be used to treat eye conditions characterized by abnormal cell proliferation, wherein the eye conditions include, but are not limited to, ocular neovascular disease, diabetic retinopathy, retinopathy of prematurity, macular degeneration, corneal graft rejection, neovascular glaucoma, retrolental fibroplasias, epidemic keratoconjunctivitis, contact lens overwear, atopic keratitis, superior limbic keratitis, pterygium keratitis sicca, myopia, chronic retinal detachment, optic pits, Terrien's marginal degeneration, hyperviscosity syndromes, chronic uveitis, chronic vitritis, presumed ocular histoplasmosis, retinitis, choroiditis, proliferative vitreoretinopathy, scleritis, Eales' disease, Best's disease, trachoma, or post-laser complications.

The present invention can be used to treat inflammatory or immune mediated diseases characterized by abnormal cell proliferation, wherein the inflammatory or immune mediated diseases include, but are not limited to, rheumatoid arthritis, osteoarthritis, ulcerative colitis, Crohn's disease, Mooren's ulcer, arthritis, sarcoidosis, inflammatory or immune mediated bowel disease, systemic lupus, Wegener's syndrome, Stevens-Johnson disease, Behcet's disease, pemphigoid, Lyme's disease, asthma or acquired immune deficiency syndrome.

The present invention can be used to treat infectious diseases characterized by abnormal cell proliferation, wherein the infectious diseases include, but are not limited to syphilis, bacterial infections, Mycobacterial infections, bacterial ulcers, fungal ulcers, Herpes simplex infections, Herpes zoster infections, protozoan infections, Bartonellosis infections, or toxoplasmosis.

The present invention can also be used to treat blood or blood vessel diseases characterized by abnormal cell proliferation, wherein the blood or blood vessel diseases include, but are not limited to, vein occlusion, artery occlusion, carotid obstructive disease, polyarteritis, atherosclerosis, Osler-Weber-Rendu disease, sickle cell anemia, leukemia, acute or chronic neoplastic disease of the bone marrow, hemangiomas, hereditary hemorrhagic telangiectasia, disease of the bone marrow, anemia, impaired blood clotting or enlargement of the lymph nodes, liver, or spleen. The present invention can also be used to treat chronic neoplastic disease of the bone marrow, wherein those diseases include, but are not limited to, multiple myeloma and myelo dysplastic syndrome.

The present invention can be used to treat skin conditions in a humans or an animals, wherein the skin conditions include, but are not limited to, abnormal wound healing, acne rosacea, chemical burns of the skin, dermatitis or psoriasis.

In addition, the invention can be used to treat a variety of post-menopausal symptoms, osteoporosis, cardiovascular disease, myocardial angiogenesis, plaque neovascularization, hemophiliac joints, angiofibroma, wound granulation, intestinal adhesions, scleroderma, hypertrophic scan; i.e., keloids. Additionally, the compounds of the present invention can be used to treat endometriosis.

In particular embodiments, one may test the inhibitors by measuring their ability to inhibit the growth of cancer cells, to induce cytotoxic events in cancer cells, to induce apoptosis of the cancer cells, to reduce tumor burden and to inhibit metastases. For example, one can measure inhibition of growth by using the MTT assay. A significant inhibition in growth is represented by decreases of at least about 30% to 40% as compared to uninhibited, and most preferably, of at least about 50%, with more significant decreases also being possible. Growth assays as measured by the MTT assay are well known in the art, for example, Mosmann, et al., 1983; Rubinstein, et al., 1990.

The compositions and methods provided herein will be described in greater detail by way of specific examples. The following examples are offered for illustrative purposes, and are intended neither to limit nor define the invention in any manner.

### EXAMPLE 1

### The Synergic Combination of ThAcr2 and X-Rays in Human Glioblastoma Cells

In addition to significantly inhibiting growth of cells with high telomerase expression as a single drug, the results obtained with ThAcr2 demonstrates that the exposure to G-quadruplex ligands targeting telomeres may radiosensitize human glioblastoma multiforme cells. The biological effect of ThAcr2 on glioblastoma cells was studied on the (IBM cell lines SF 763 and SF 767 established in the Brain Tumor Research Center Tissue Bank at the University of California, San Francisco. The GBM cell lines were examined for telomere status. SF 763 and SF 767 cells displayed relatively short telomeres (mean telomere restriction fragment length of 4.4 kb and 3.3 kb, respectively) and strongly expressed telomerase as measured using real-time RT-PCR for the human telomerase reverse transcriptase gene (hTERT) mRNA.

(a) ThAcr2 as a Growth Inhibitor.

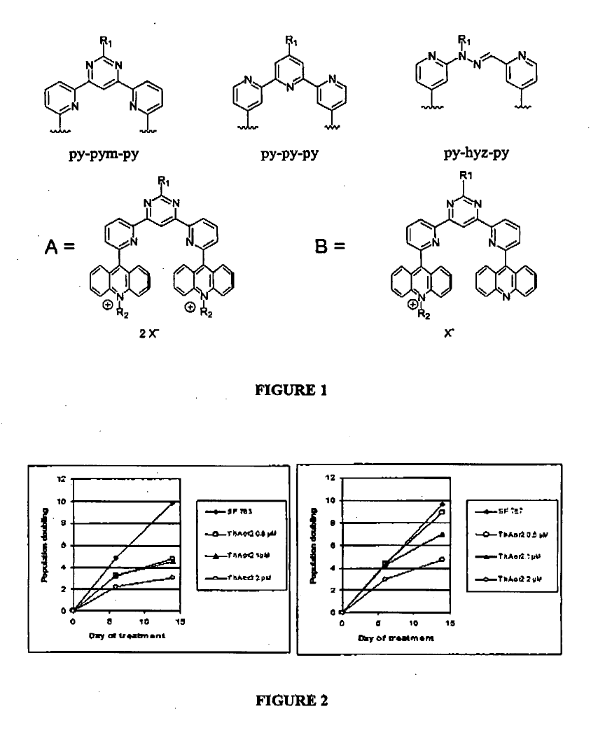

GBM cells were maintained in culture for 14 days in the presence of 0.5, 1 and 2 μM ThAcr2. This continuous treatment inhibited tumor cell growth in both cell lines with a more pronounced effect on SF 763 cells (FIG. 1). Analysis of short-term (3 days) cell viability indicated that the concentrations of ThAcr2 (0.5, 1 and 2 μM) were non-toxic.

(b) ThAcr2's Effect on Telomeres.

The effects of 1 μM ThAcr2 on telomere and telomerase after 7 and 14 days of treatment were evaluated. The analysis revealed that the treatment did not reduce telomere length Tumor cells treated with ThAcr2 exhibited a 3- to 5-fold increase in hTERT expression, depending on the cell line. However, cytogenetic analysis performed at day 7 documented the presence of multiple telomere fusions in ThAcr2-treated cells. The results suggest that ThAcr2 induces an alteration in telomere-capping function, independently of telomere shortening or the level of telomerase expression.

(c) ThAcr2's Effect on Cell Cycle.

Using flow cytometry, cell cycle distribution and apoptosis in cells treated for 7 days with 0.5-2 μM ThAcr2 were examined. Compared to untreated controls, cells exposed to ThAcr2 showed a moderate accumulation in the O₂/M phase of cell cycle. The percentage of cells in the G2/M phase increased from 10% to 20%. The annexin-propidium iodide (apoptotic) fraction was essentially identical (9-11%) in control and treated cells at lower ThAcr2 concentrations (0, 5 and 1 μM) but increased to 34% in cells treated with 2 μM ThAcr2. These results show that low concentrations of ThAcr2 (0.5 and 1 μM) did not significantly perturb the cell cycle and did not induce apoptosis of GBM cells.

(d) Long-Term Treatment with ThAcr2.

To further investigate the tumor-growth inhibition effect of ThAcr2 as a single agent, the GBM cells were exposed to 1 μM ThAcr2 for 50 days. In both GBM cell lines, an increase in the rate of cell proliferation was observed from day 30, which was associated with an overexpression of telomerase (up to 7-fold for SF 763 cells). The development of resistance to ThAcr2 after long-term treatment suggests that the exposure to the ligand should be short-term and combined with established cytotoxic agents to improve anti-tumor effect.

(e) ThAcr2 in Combination with Ionizing Radiations.

To explore the effect of ThAcr2 in combination with ionizing radiation on GBM cells, clonogenic survival of SF 763 and SF 767 cells was analyzed. Although both cell lines were radio-resistant, the surviving fraction after 2 Gy was greater in SF 763 (80%) than in SF 767 cells (56%), indicating stronger resistance of SF 763 cells to radiation.

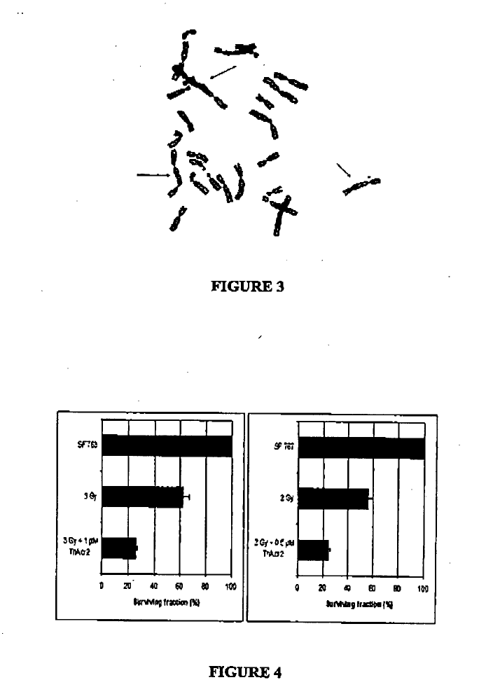

For combination experiments, non-toxic sub-apoptotic ThAcr2 concentrations of 0.5 and 1 μM were utilized. The duration of treatment corresponded to earliest time-points where ThAcr2 showed an appreciable anti-proliferative effect in the GBM cell lines. SF 763 cells were treated with 1 M ThAcr2 for 7 days, then replated and irradiated with a 3 Gy-dose of X-rays. SF 767 cells were treated with 0.5 μM ThAcr2 for 14 days, then replated and irradiated with a 2-Gy dose. After correction for the suppressive effect on colonies of ThAcr2 alone, ThAcr2 significantly reduced the surviving fraction of tumor cells 2.5 fold in the SF 763 line and 2.3 fold in the SF 767 line (FIG. 3). The combination of ThAcr2 with radiation was additive when the ThAcr2 treatment started after irradiation. The combination of ThAcr2 with radiation was supra-additive or had a synergistic effect, when GBM tumor cells were continuously treated before and after irradiation.

Thus, the present invention demonstrates the effect in treating a disease characterized by abnormal cell proliferative using a therapeutic combination comprising ThAcr2, or pharmaceutically acceptable salts thereof, before and after radiation, and further that the treatment radio-sensitizes human GBM cells.

### EXAMPLE 2

### Combination Therapies Comprising ThAcr2 and Other Chemotherapeutic Agents

Chemotherapeutic agents contemplated to be of use with the present invention include, but are not limited to, adriamycin, 5-fluorouracil (5-FU), etoposide (VP-16), camptothecin, actinomycin-D, mitomycin, cisplatin (CDDP), hydrogen cyclophosphamide, ifosfamide, melphalan, chlorambucil, bisulfan, nitrosurea, dactinomycin, daunorubicin, doxorubicin, bleomycin, plicomycin, tamoxifen, taxol, transplatinum, vincristin, vinblastin, and methotrexate. Chemotherapeutic agents capable of inducing damage to DNA include compounds that interfere with the replication, mitosis, or chromosomal segregation of DNA. Such agents include, but are not limited to, adriamycin, doxorubicin, etoposide, verapamil, and podophyllotoxin. In the combination therapy contemplated by the present invention, one would generally contact the target cells with the novel compounds of the present invention and at least one other chemotherapeutic agent mentioned herein. The combination of the novel compounds and at least one other chemotherapeutic agent would be administered in an effective amount sufficient to kill the cell, or inhibit the proliferation of the cell. This combination treatment may also be achieved by contacting the target cells with a single composition or pharmacological formulation that includes both agents, or by contacting the target cells with one or more distinct compositions or formulations, either sequentially, simultaneously or intermittently.

### EXAMPLE 3

### Combination Therapies Comprising ThAcr2 and Radiosurgery

Surgery is often combined with radiotherapy or chemotherapy to ensure the destruction of any remaining neoplastic or malignant cells. Localization and surgical removal of a tumor is contemplated to be used sequentially with in vivo treatment of the patient with an effective amount of ThAcr2. In addition, radiotherapy before and after surgical removal of a tumor, in combination with in vivo treatment of the patient with an effective amount of ThAcr2 in contemplated by the present invention.

Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

### REFERENCES

A number of patents and publications are cited above in order to more fully describe and disclose the invention and the state of the art to which the invention pertains. Full citations of these references are listed below. Each of these references are incorporated herein by reference in its entirety into the present disclosure.
Alberti, et al., 2002, “Benzoindoloquinolines Interact with DNA Tetraplexes and Inhibit Telomerase,” Biorganic & Medicinal Chemistry Letters, Vol. 12, pp. 1071-1074.
Allshire, et al., 1988, “Telomeric repeat from T. thermophilia cross hybridizes with human telomeres,” Nature, 332:656-659.
Autexier, 1999, “Telomerase as a Possible Target for Anticancer Therapy,” Chemistry & Biology, November 1999, Vol. 6, pp. R299-R303.
Blackburn, 1991, “Structure and function of telomeres,” Nature, 350:569-573.
Blackburn, et al., Eds., 1995, In: Telomeres,” Cold Spring Harbor Press, New York.
Bostock-Smith, et al., 1999, “Molecular Recognition between a New Pentacyclic Acridinium Salt and DNA Sequences Investigated by Optical Spectroscopic Techniques, Proton Nuclear Magnetic Resonance Spectroscopy, and Molecular Modeling,” Biochemistry, Vol. 38, No. 21, pp. 6723-6731.
Brem, et al., 1991, S, Neurosurg. 14:411-446.
Broccoli, et al., 1994, “Telomerase activity in normal and malignant hematopoietic cells,” Proc. Natl. Acad. Sci. U.S.A., 92:9082-9086.
Carrasco, et al., 2002, “Tight Binding of the Antitumour Drug Ditercalcinium to Quaduplex DNA,” Chem Bio Chem, Vol. 3, pp. 1235-1241.
Corey, 2002, “Telomerase Inhibition, Oligonucleotides, and Clinical Trials,” Oncogene, Vol. 21, pp. 631-637.
Counter, et al., 1992, “Telomere shortening associated with chromosome instability is arrested in immortal cells which express telomerase activity,” EMBO J. 11:1921-1929.
Dimri, et al., 1995, Proc. Natl. Acad. Sci. USA 92:20.
Feng, et al., 1995, “The RNA component of human telomerase,” Science, 269:1236-1241.
Gimenez-Amau, et al., 1998a, “Antitumour Polycyclic Acridines, Part 2,” Anti-Cancer Drug Design, Vol. 13, pp. 125-143.
Gimenez-Amau, et al., 1998b, “Antitumour Polycyclic Acridines, Part 4,” Anti-Cancer Drag Design, Vol. 13, pp. 431-451.
Gomez, et al., 2002, “Detection of Telomerase Inhibitors Based on O-Quadruplex Ligands by a Modified Telomeric Repeat Amplification Protocol Assay,” Cancer Research, Vol. 62, pp. 3365-3368.
Gowan, et al., 2002, “A G-Quadruplex-Interactive Potent Small-Molecule Inhibitor of Telomerase Exhibiting in Vitro and in Vivo Antitumour Activity,” Molecular Pharmacology, Vol. 61, No. 5, pp. 1154-b 1162.
Hagan, et al., 1997, “Antitumour Polycyclic Acridines, Part 1,” J. Chem. Soc., Perkin Trans. 1, pp. 2739-2746.
Hagan, et al., 1998, “Antitumour Polycyclic Acridines, Part 3,” J. Chem. Soc., Perkin Trans. 1, p. 915-923.
Harley, et al., 1990, “Telomeres shorten during aging of human fibroblasts,” Nature, 345:458-460.
Harley, et al., 1994, “Telomerase, Cell Immortality, and Cancer,” Cold Spring Harbor Syrup. Quant Biol., 59:307-315.
Harrison, et al., 1999, “Human Telomerase Inhibition by Substituted Acridine Derivatives,” Bioorganic & Medicinal Chemistry Letters, Vol. 9, pp. 2463-2468.
Herber, et al., 2001, “Telomerase and Breast Cancer,” Breast Cancer Research, Vol. 3, pp. 146-149.
Hiyama, et al., 1995a, “Telomerase activity in small-ell and non-small-ell lung cancers,” Natl. Cancer Inst., 87:895-902.
Hiyama, et al., 1995b, “Correlating telomerase activity levels with human neuroblastoma outcomes,” Nature Medicine, 1:249-255.
Hochhauser, 1997, AntiCancer Chemotherapeutic Agents 8:903.
Julino, et al., 1998, “Antitumour Polycyclic Acridines, Part 5,” J. Chem. Soc., Perkin Trans. 1, pp. 1677-1684.
Kern, et al., 2002, “The Relationship between Ligand Aggregation and G-Quadruplex DNA Selectivity in a Series of 3,4,9,10-Perylenetetracarboxylic Acid Diimides,” Biochemistry, Vol. 41, pp. 11379-11389.
Kim, et al., 1994, “Specific association of human telomerase activity with immortal cells and cancer,” Science, 266:2011-2015.
Kim; et al., 2002, “Telomestatin, a Potent Telomerase Inhibitor That Interacts Quite Specifically with the Human Telomeric Intramolecular G-Quadruplex,” J. Amer. Chem. Soc., Vol. 124, No. 10, pp. 2098-2099.
Liu, Rong, Ed., 2000, Water Insoluble Drug Formulation, pp. 1-633, CRC Press LLC.
Mergny, et al., 2002, “Natural and Pharmacological Regulation of Telomerase,” Nucleic Acids Research, Vol. 30, No. 4, pp. 839-865.
Mosmann, et al., 1983, “Rapid colorimetric assay for cellular growth and survival: application to proliferation and cytotoxicity assays,” J. Immunol. Methods, 65(1-2), pp. 55-63.

Neidle, et al., 1999, “Telomerase as an Anti-Cancer Target: Current Status and Future Prospects,” AntiCancer Drug Design, Vol. 14, pp. 341-347.
Neidle, et al., 2002a, “Telomere Maintenance as a Target for Anticancer Drug Discovery,” Nature Reviews, Vol. 1, May 2002, pp. 383-393.
Neidle, et al., 2002b, International Patent Application No. PCT/GB01/03046, publication number WO 02/08193 published 17 Jan. 2002.
Neidle, et al., 2005, Therapeutic Acridone and Acridine, US-2005-0070568-A1, published 31 Mar. 2005.
Norton, et al., 1996“Inhibition of human telomerase activity by peptide nucleic adds,” Nature Biotechnology, 14:615-619.
Parkinson, 1996, “Do telomerase antagonists represent a novel anti-cancer strategy?” Brit. J. Cancer, 73:14.
Parkinson, et al., 2002, “Crystal structure of parallel quadruplexes from human telomeric DNA,” Nature, Vol. 417, 20 Jun. 2002, pp. 876-880.
Patani et al., 1996, “Bio-isosterism: a rational approach in drug design, >> Chem. Rev., p. 3147-3176.
Perry, et al., 1998a, “1,4- and 2,6-Disubstituted Amidoanthracene-9,10-dione Derivatives as Inhibitors of Human Telomerase,” J. Med. Chem., Vol. 41, No. 17, pp. 3253-3260.
Perry, et al., 1998b, “Human Telomerase Inhibition by Regioisomeric Disubstitued Amidoanthracene-9,10-diones,” J. Med. Chem., Vol. 41, No. 24, pp. 4873-4884.
Perry, et al., 1998c, “Telomeres and Telomerase: Targets for Cancer Chemotherapy?,” Exp. Opin. Ther. Patents, Vol. 8, No. 12, pp. 1567-1586.
Perry, et al., 1999a, “Design, Synthesis and Evaluation of Human Telomerase Inhibitors Based Upon a Tetracyclic Structural Motif,” Anti-Cancer Drug Design, Vol. 14, pp. 373-382.
Perry, et al., 1999b, “2,7-Disubstituted Amidofluorenone Derivatives as Inhibitors of Human Telomerase,” J. Med. Chem., Vol. 42, No. 14, pp. 2679-2684.
Poele, et al., 1999, Br. J. Cancer 80:9.
Riou, et al., 2002, “Cell Senescence and Telomere Shortening Induced by a New Series of Specific G-Quadruplex DNA Ligands,” Proc. Nat. Acad. Sci., Vol. 99, No 5, pp. 2672-2677.
Rhyu, 1995, “Telomeres, telomerase, and immortality,” Natl. Cancer Inst., 87:884-894.
Rubinstein, et al., 1990, “Comparison of in vitro anticancer-drug-screening data generated with a tetrazolium assay versus a protein assay against a diverse panel for human tumor cell lines,” J. Natl. Cancer Inst., 82(13), pp. 1113-1118.
Sharma, et al., 1997, “Preclinical and Clinical Strategies for Development of Telomerase and Telomere Inhibitors,” Annals of Oncology, Vol. 8, pp. 1063-1074.
Shay, et al., 2002, “Telomerase: A Target for Cancer Therapeutics,” Cancer Cell, Vol. 2, pp. 257-265.
Sun, et al., 1997, “Inhibition of Human Telomerase by a O-Quadruplex-Interactive Compound,” J. Med. Chem., Vol. 40, pp. 2113-2116.
Urquidi, et al., 1998, “Telomerase in Cancer: Clinical Applications,” Ann. Med., Vol. 30, pp. 419-430.
Wang, et al., 1998, Cancer Res. 58:5019.
Yamamoto, et al., 1982, Bull. Chem. Soc. Jpn. 30, 1731-1737.

What is claimed is:

1. A composition containing a spacer structure selected from: 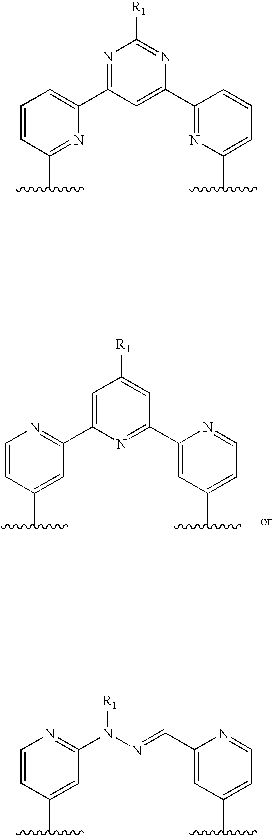
wherein the composition with the spacer structure is a hydrocarbon and
wherein R₁ is a hydrogen, an aliphatic or an aromatic substituent.

2. The composition of claim 1, wherein the composition has the 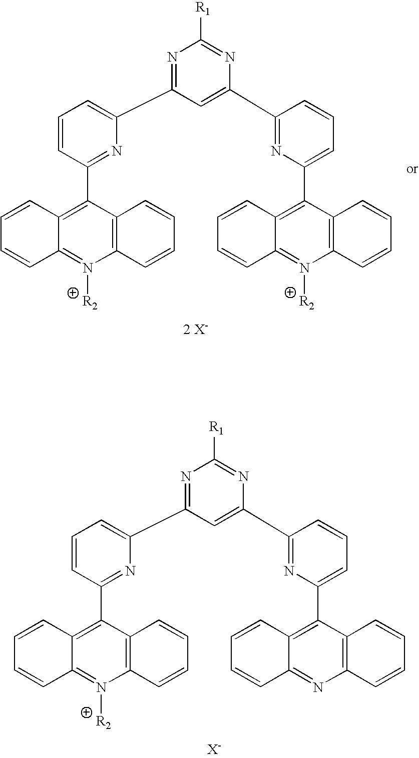
wherein R₁ is selected from the group comprising a hydrogen or an aliphatic, an aromatic, a hydrophilic, a hydrophobic, a hydrophilic, a cyclic, or an acyclic substituent, and wherein R₂ is an alkyl, a hydrophilic, or a hydrophobic substituent; and wherein X⁻ (or 2X⁻) is selected from the group consisting of triflate, chloride, bromide, iodide, nitrate, or sulphate.

3. A composition comprising:

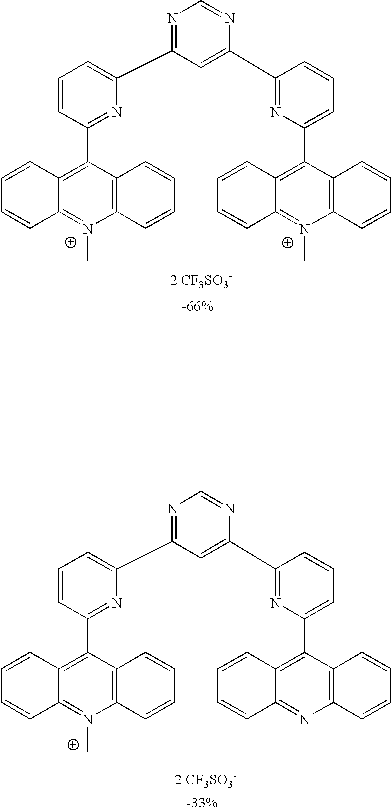

4. The pharmaceutically acceptable salts, esters, amides, solvates, hydrates, and protected forms of the composition of claim 1.

5. The composition of claim 1, wherein the composition is in the form of particles and wherein the average particle size is less than about 1900 nm.

6. The composition of claim 1, wherein the composition is in the form of particles and wherein the average particle size is less than about 1500 nm.

7. The composition of claim 1, wherein the composition is in the form of particles and wherein the average particle size is less than about 500 nm.

8. A method of treating abnormal cell proliferation in a mammal comprising administering to the mammal an effective amount of a composition containing a spacer structure selected from: 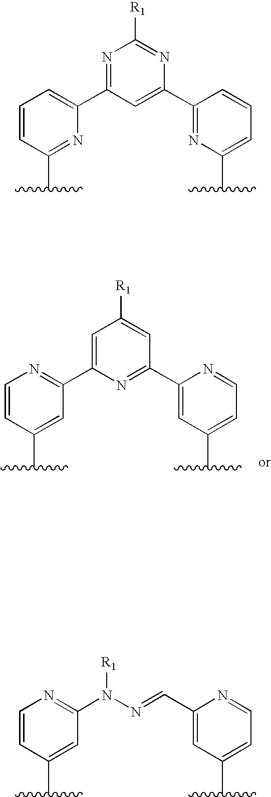
wherein the composition with the spacer structure is a hydrocarbon and
wherein R₁ is a hydrogen, an aliphatic or an aromatic substituent.

9. The method of claim 8, wherein the composition has the structure: 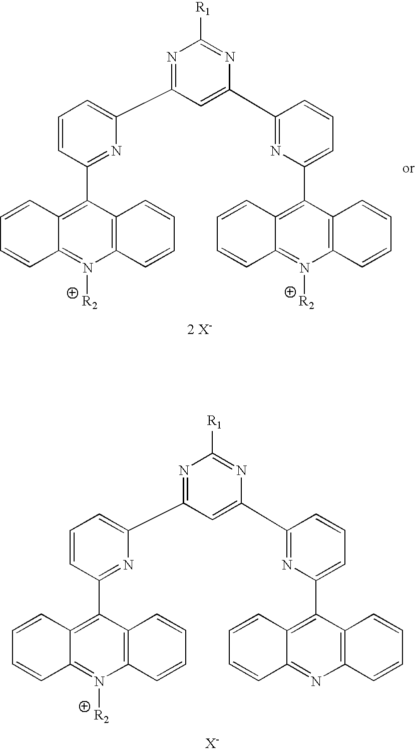
wherein R₁ is selected from the group comprising a hydrogen or an aliphatic, an aromatic, a hydrophilic, a hydrophobic, a hydrophilic, a cyclic, or an acyclic substituent; and wherein R₂ is an alkyl, a hydrophilic, or a hydrophobic substituent; and wherein X⁻ (or 2X⁻) is selected from the group consisting of triflate, chloride, bromide, iodide, nitrate, or sulphate.

10. The method of claim 9, wherein the composition is:

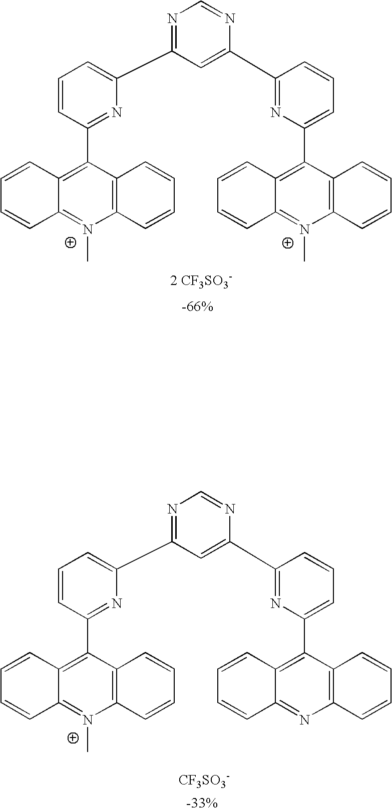

11. The method of claim 8, wherein the administration of the composition is in a daily dose, a daily sub-dose, or any appropriate fraction thereof to the human or animal.

12. The method of claim 8, wherein the amount of the composition administered is approximately 0.1 to approximately 300 mg/kg/day.

13. The method of claim 8, wherein the administration of the composition is oral, parenteral, transdermal, topical, intravenous, subcutaneous, intramuscular, intradermal, ophthalmic, epidural, intratracheal, sublingual, buccal, rectal, vaginal, nasal or inhalation.

14. The method of claim 8, wherein the composition further comprises an additive selected from an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof.

15. The method of claim 8, wherein the composition is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

16. The method of claim 8, wherein the abnormal cell proliferation is associated with cancers selected from brain cancer, breast cancer, cancers of the head, neck or larynx, prostrate cancer, renal cell cancer, ovarian cancer, colon cancer, bladder cancer, pancreatic cancer, stomach cancer, esophageal cancer, cutaneous melanoma, liver cancer, lung cancer, testicular cancer, kidney cancer, bladder cancer, cervical cancer, lymphoma, parathyroid cancer, penile cancer, rectal cancer, anal cancer, small intestine cancer, thyroid cancer, uterine cancer, Hodgkin's lymphoma, lip and oral cancer, skin cancer, leukemia, spinal cancer, vaginal cancer, vulva cancer, soft tissue sarcomas, lymphoma or multiple myeloma.

17. The method of claim 16, wherein the brain cancer is glioblastoma multiforme.

18. The method of claim 8, wherein the abnormal cell proliferation is associated with diabetic retinopathy, retinopathy of prematurity, corneal graft rejection, neovascular glaucoma, retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, arthritis, rheumatoid arthritis, polyarteritis, systemic lupus, Wegener's sarcoidosis, scleritis, Stevens-Johnson disease, radial keratotomy, macular degeneration, sickle cell anemia, sarcoid, pseudoxanthoma elasticum, Paget's disease, vein occlusion, artery occlusion, carotid obstructive disease, chronic uveitis, chronic vitritis, Lyme's disease, Eales' disease, Behcet's disease, myopia, optic pits, Stargardt's disease, pars planitis, chronic retinal detachment, hyperviscosity syndromes, toxoplasmosis, post-laser complications, abnormal proliferation of fibrovascular or fibrous tissue, hemangiomas, Osler-Weber-Rendu disease, solid tumors, blood-borne tumors, benign tumors, acquired immune deficiency syndrome, ocular neovascular disease, age-related macular degeneration, osteoarthritis, diseases caused by chronic inflammation, Crohn's disease, ulcerative colitis, tumors of rhabdomyosarcoma, tumors of retinoblastoma, Ewing's sarcoma, neuroblastoma, tumors of osteosarcoma, leukemia, psoriasis, atherosclerosis, pemphigoid, infections causing retinitis, infections causing choroiditis, presumed ocular histoplasmosis, Best's disease, proliferative vitreoretinopathy, Bartonellosis, acoustic neuromas, neurofibroma, trachoma, pyogenic granulomas, vascular malfunctions, abnormal wound healing, gout or gouty arthritis, angiogenesis-dependent cancer, hereditary hemorrhagic telangiectasia, post-menopausal symptoms, osteoporosis, cardiovascular disease, myocardial angiogenesis, plaque neovascularization, hemophiliac joints, angiofibroma, wound granulation, intestinal adhesions, scleroderma, keloids, endometriosis.

19. A method of inhibiting telomerase activity in a mammal comprising administering to the mammal an effective amount of a composition containing a spacer structure selected from: 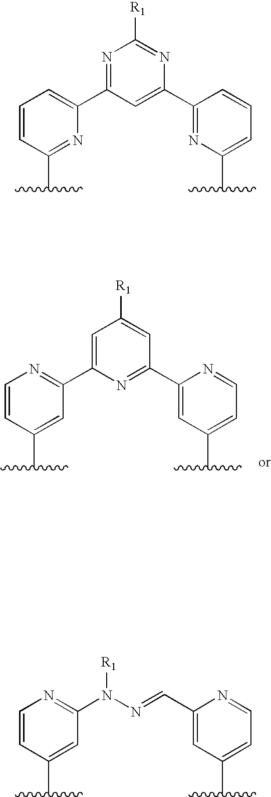
wherein the composition with the spacer structure is a hydrocarbon and
wherein R₁ is a hydrogen, an aliphatic or an aromatic substituent.

20. The method of claim 19, wherein the composition has the structure: 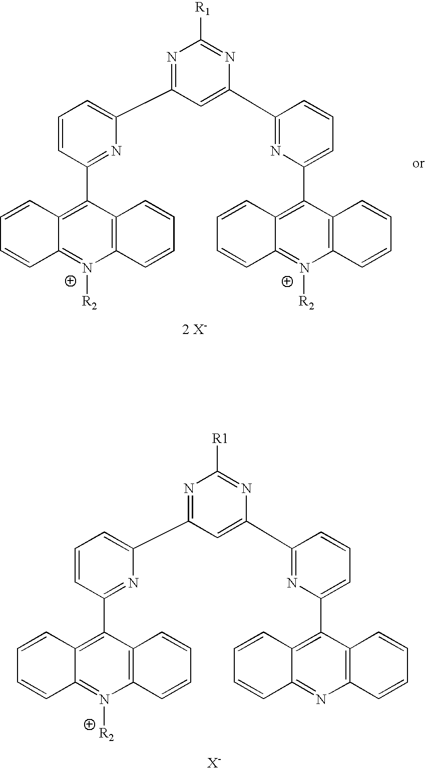
wherein R₁ is selected from the group comprising a hydrogen or an aliphatic, an aromatic, a hydrophilic, a hydrophobic, a hydrophilic, a cyclic, or an acyclic substituent; and wherein R₂ is an allyl, a hydrophilic, or a hydrophobic substituent; and wherein X⁻ (or 2X⁻) is selected from the group consisting of triflate, chloride, bromide, iodide, nitrate, or sulphate.

21. The method of claim 20, wherein the composition is:

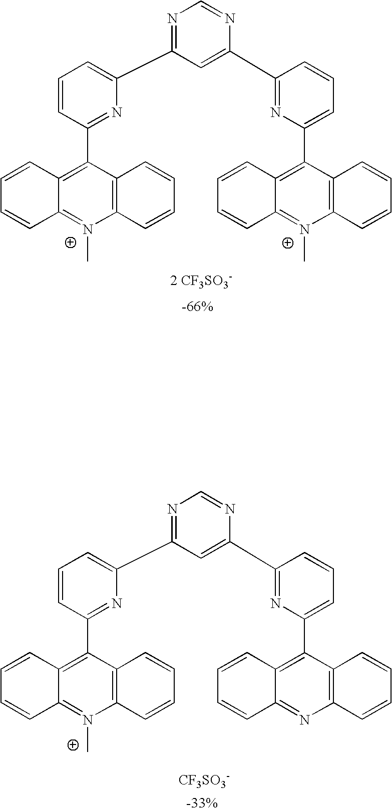

22. A pharmaceutical preparation comprising
(a) a composition containing a spacer structure selected from: 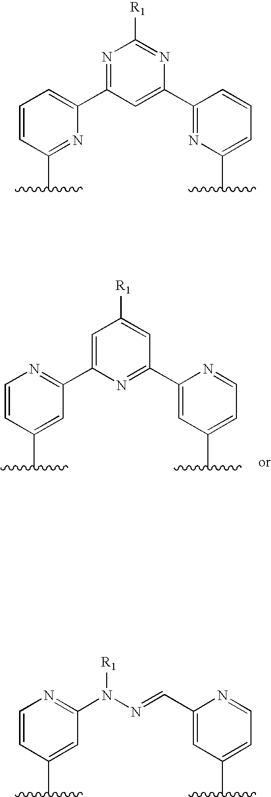
wherein the composition with the spacer structure is a hydrocarbon and wherein R₁ is a hydrogen, an aliphatic or an aromatic substituent; and
(b) a pharmaceutically acceptable carrier, excipient or diluent.

23. The pharmaceutical preparation of claim 22, wherein
(a) the composition has the structure: 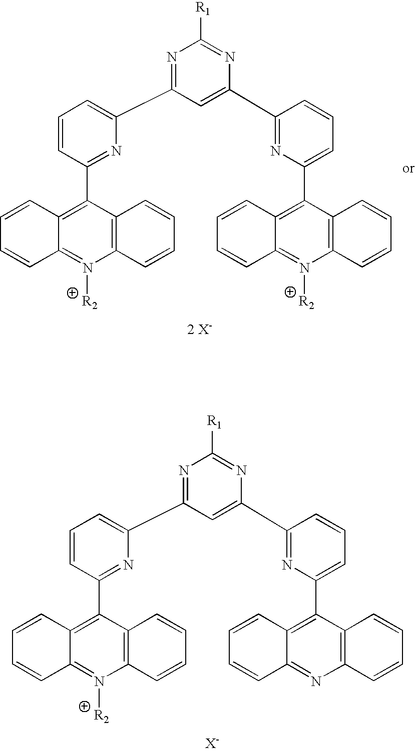
wherein R₁ is selected from the group comprising a hydrogen or an aliphatic, an aromatic, a hydrophilic, a hydrophobic, a hydrophilic, a cyclic, or an acyclic substituent; and wherein R₂ is an alkyl, a hydrophilic, or a hydrophobic substituent; and wherein X⁻ (or 2X⁻) is selected from the group consisting of triflate, chloride, bromide, iodide, nitrate, or sulphate; and
(b) a pharmaceutically acceptable carrier, excipient or diluent.

24. The pharmaceutical preparation of claim 23, wherein the composition is:

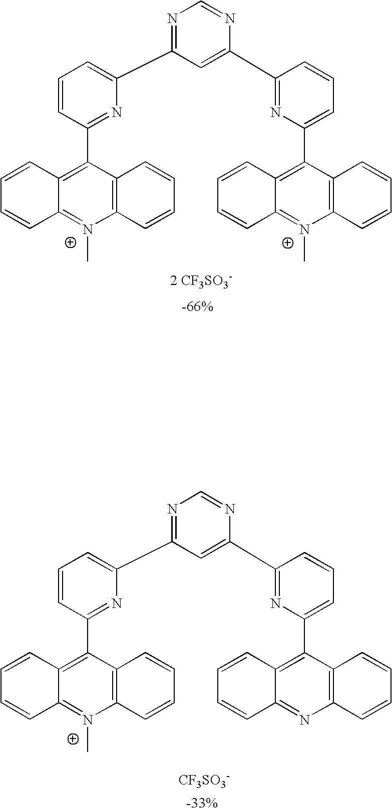

25. A therapeutic combination comprising:
(a) administering to a mammal an effective amount of a composition containing a spacer structure selected from 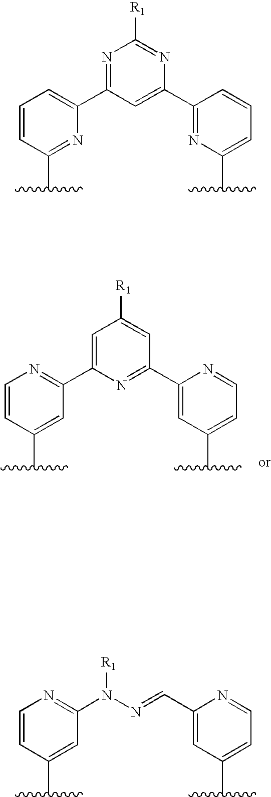
wherein the composition with the spacer structure is a hydrocarbon and wherein R₁ is a hydrogen, an aliphatic or an aromatic substituent; and
(b) the simultaneous, separate or sequential administration, in synergistically effective amounts, of at least one of radiotherapy, radiosurgery, or radiotherapy.

26. The therapeutic combination of claim 25, wherein the composition has the structure: 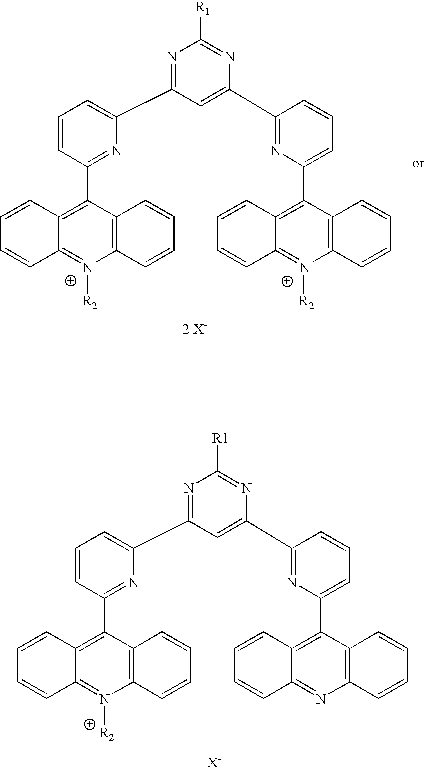
wherein R₁ is selected from the group comprising a hydrogen or an aliphatic, an aromatic, a hydrophilic, a hydrophobic, a hydrophilic, a cyclic, or an acyclic substituent; and wherein R₂ is an alkyl, a hydrophilic, or a hydrophobic substituent and wherein X⁻ (or 2X⁻) is selected from the group consisting of triflate, chloride, bromide, iodide, nitrate, or sulphate.

27. The therapeutic combination of claim 26, wherein the composition is:

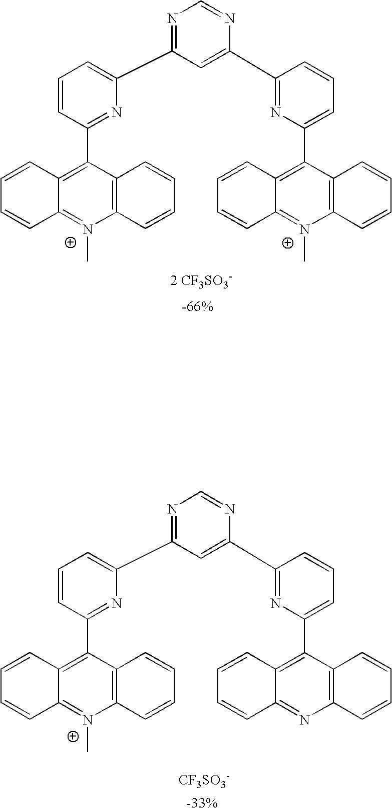

28. The therapeutic combination of claim 25, further comprising at least one radiomimetic agent selected from the group consisting of bleomycin, neocarzinostatin, adriamycin, mitomycin C, streptonigrin, methylmethanesulfonate and enediynes.

29. The therapeutic combination of claim 25, wherein the radiotherapy is at least one of external radiation, internal radiation, systematic radiation, hyperthermia, radio-immunotherapy, photodynamic therapy, X-ray radiation, gamma-ray radiation, or particle beams.

30. A therapeutic combination comprising:
(a) administering to a mammal an effective amount of a composition containing a spacer structure selected from 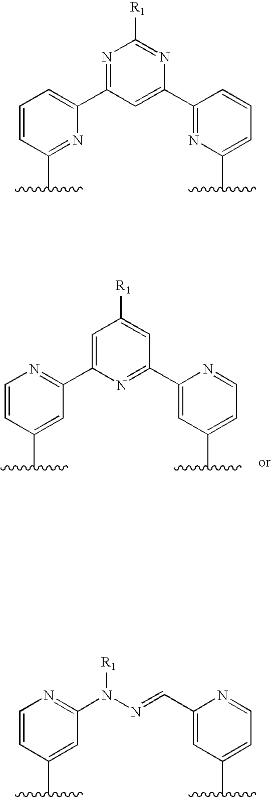
wherein the composition with the spacer structure is a hydrocarbon and wherein R₁ is a hydrogen, an aliphatic or an aromatic substituent; and
(b) the simultaneous, separate or sequential administration of at least one other chemotherapeutic agent.

31. The therapeutic combination of claim 30, wherein the composition has the structure: 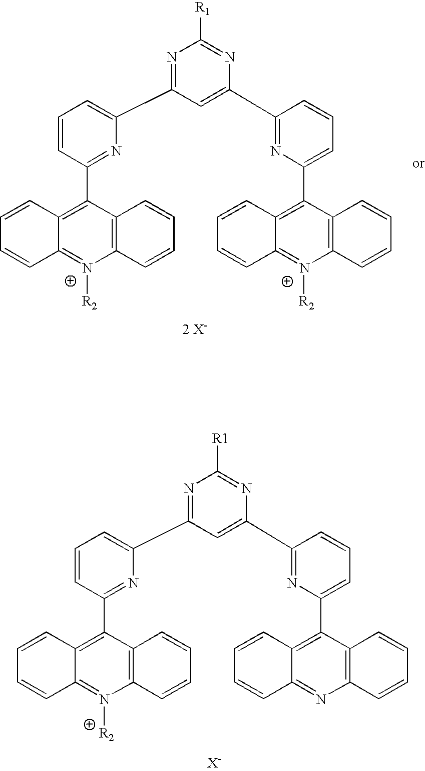
wherein R₁ is selected from the group comprising a hydrogen or an aliphatic, an aromatic, a hydrophilic, a hydrophobic, a hydrophilic, a cyclic, or an acyclic substitutent; and wherein R₂ is an alkyl, a hydrophilic, or a hydrophobic substituent; and wherein X⁻ (or 2X⁻) is selected from the group consisting of triflate, chloride, bromide, iodide, nitrate, or sulphate.

32. The therapeutic combination of claim 31, wherein the composition is: [chemical structure]
